(19) United States
(12) Patent Application Publication
(10) Pub. No.: US 2022/0151674 A1
(43) Pub. Date: May 19, 2022

(54) Systems and Methods for Selective Tissue Ablation

(71) Applicant: Santa Anna Tech LLC, Santa Ana, CA (US)

(72) Inventor: Virender K. Sharma, Paradise Valley, AZ (US)

(21) Appl. No.: 17/455,537

(22) Filed: Nov. 18, 2021

Related U.S. Application Data
(60) Provisional application No. 63/115,389, filed on Nov. 18, 2020.

(51) Int. Cl. A61B 18/04

(52) U.S. Cl. CPC A61B 18/04; A61B 2018/00577

(57) ABSTRACT

Ablation catheters and systems include catheter tips with a positioning element to ablate a target tissue that damages substantial cellular component in the target tissue while avoiding significant damage to the extra cellular matrix.

### CROSS-REFERENCE

The present application relies on, for priority, U.S. Patent Provisional Application No. 63/115,389, titled “Systems and Methods for Selective Tissue Ablation” and filed on Nov. 18, 2020, which is herein incorporated by reference in its entirety.

### FIELD

The present specification relates to systems and methods configured to generate and deliver vapor for ablation. More particularly, the present specification relates to systems and methods comprising a vapor ablation catheter and vapor generation for delivering differential ablation to the cellular structures and to the extra cellular matrix, of a tissue.

### BACKGROUND

The extracellular matrix (ECM) is a three-dimensional network of extracellular macromolecules, such as collagen, enzymes, and glycoproteins, that provide structural and biochemical support to surrounding cells. Multicellularity evolved independently in different multicellular lineages, therefore the composition of ECM varies between multicellular structures. However, cell adhesion, cell-to-cell communication and differentiation are common functions of the ECM.

Due to its diverse nature and composition, the ECM can serve many functions, such as providing support, segregating tissues from one another, and regulating intercellular communication. The extracellular matrix regulates a cell's dynamic behavior. In addition, it sequesters a wide range of cellular growth factors and acts as a local store for them. Changes in physiological conditions can trigger protease activities that cause local release of such stores. This allows the rapid and local growth factor-mediated activation of cellular functions without de novo synthesis. Formation of the extracellular matrix is essential for processes like growth, wound healing, and fibrosis. An understanding of ECM structure and composition also helps in comprehending the complex dynamics of tumor invasion and metastasis in cancer biology as metastasis often involves the destruction of ECM by enzymes such as serine proteases, threonine proteases, and matrix metalloproteinases.

ECM has been found to cause regrowth and healing of tissue. Although the mechanism of action by which ECM promotes constructive remodeling of tissue is still unclear, researchers now believe that matrix-bound nanovesicles (MBVs) are a key player in the healing process. In human fetuses, for example, the ECM works with stem cells to grow and regrow all parts of the human body, and fetuses can regrow anything that gets damaged in the womb. Scientists have long believed that the matrix stops functioning after full development. It has been used in the past to help horses heal torn ligaments, but it is being researched further as a device for tissue regeneration in humans.

In terms of injury repair and tissue engineering, the ECM serves two main purposes. First, it prevents the immune system from triggering from the injury and responding with inflammation and scar tissue. Next, it facilitates the surrounding cells to repair the tissue instead of forming scar tissue.

Ablation, as it pertains to the present specification, relates to the removal or destruction of a body tissue, via the introduction of a destructive agent, such as radiofrequency energy, laser energy, ultrasonic energy, cyroagents, heated vapor and/or steam. Ablation is commonly used to eliminate diseased or unwanted tissues, such as, but not limited to cysts, polyps, tumors, hemorrhoids, and other similar lesions. Ablation techniques may be used in hyperthermia in combination with chemotherapy, radiation, surgery, and Bacillus Calmette-Guérin (BCG) vaccine therapy, among others.

Steam-based ablation systems, such as the ones disclosed in U.S. Pat. Nos. 9,615,875, 9,433,457, 9,376,497, 9,561,068, 9,561,067, and 9,561,066, disclose ablation systems that controllably deliver steam through one or more lumens toward a tissue target. One problem that all such steam-based ablation systems have is the potential overheating or burning of healthy tissue. Steam passing through a channel within a body cavity heats up surfaces of the channel and may cause exterior surfaces of the medical tool, other than the operational tool end itself, to become excessively hot. As a result, physicians may unintentionally burn healthy tissue when external portions of the device, other than the distal operational end of the tool, accidentally contacts healthy tissue. U.S. Pat. Nos. 9,561,068, 9,561,067, and 9,561,066 are incorporated herein by reference.

It is desirable to selectively ablate the cellular elements of the tissue without significantly ablating the ECM, allowing for the tissue to heal adequately after an ablation procedure without resulting in a complication, such as bleeding or stricture formation. It is also desirable to selectively ablate tumor cells of the tissue without significantly ablating regular or normal cells and ECM. It is therefore desirable to have steam-based ablation devices that integrate into the device itself safety mechanisms which prevent unwanted ablation during use.

### SUMMARY

The present specification discloses a method for selectively ablating at least one of a target tissue area of a patient, the method comprising: providing an ablation system comprising: at least one pump; a coaxial catheter for inserting into the patient, the coaxial catheter comprising: an outer catheter for advancing to the target tissue of the patient; an inner catheter for advancing into the target tissue of the patient, concentric and slidable within the outer catheter, wherein the inner catheter is in fluid communication through a catheter connection port with the at least one pump, wherein a proximal end of the inner catheter is connected to the catheter connection port to place the inner catheter in fluid communication with the at least one pump, wherein the inner catheter comprises: at least one lumen to transport an ablative agent delivered from the at least one pump; at least one electrode positioned within the at least one lumen; at least one positioning element along a length of the inner catheter; and at least one opening proximate to the positioning element of the inner catheter; a controller having at least one processor in data communication with the at least one pump, wherein, upon activating, the controller is configured to: control the delivery of the ablative agent into the at least one lumen in the coaxial catheter; control the delivery of an electrical current to the at least one electrode positioned within the at least one lumen of the inner catheter; and control vapor generated from the ablative agent; inserting the coaxial catheter into the target tissue of the patient; applying the positioning element proximate the target tissue area enclosing at least a portion of the target tissue; and programming the controller to control a delivery of the vapor such that the target tissue is ablated to cause differential damage to different cellular components in the target tissue.

Optionally, the at least one positioning element is advanced until the distal end of the positioning element encloses the target tissue area.

Optionally, the at least one positioning element is advanced until the distal end of the positioning element is proximate the target tissue area.

Optionally, programming the controller to control a delivery of the vapor such that the target tissue is ablated to cause differential damage comprises damaging more cellular structure relative to extra cellular matrix (ECM). The target tissue may be ablated for a time period at a temperature of up to 60° C. Greater than 50% of the cellular structure may undergo irreversible damage and less than 50% of the ECM may be damaged.

Optionally, programming the controller comprises maintaining pressure at the target tissue area less than 5 atm.

Optionally, programming the controller comprises delivering the vapor at a temperature between 99° C. and 110° C.

Optionally, programming the controller comprises delivering the vapor of a quality greater than 25%.

Optionally, programming the controller to control a delivery of the vapor such that the target tissue is ablated to cause differential damage comprises damaging more cellular structure relative of tumor relative to normal cellular structure.

Optionally, the method further comprises treating a tumor proximate one of a blood vessel and a bowel wall.

Optionally, the method further comprises performing trans-arterial vapor ablation of tumors. Optionally, the method comprises providing the ablation system positioned within a hepatic artery that feeds a tumor in a liver.

Optionally, the method further comprises treating pain in at least one of a back, a neck, a sacroiliac joint, a knee pain, and a hip joint. Optionally, the method comprises treating pain transmitted by a nerve proximate a facet joint in a spinal motion segment of a patient.

Optionally, the method comprises administering vapor for basivertebral nerve ablation.

Optionally, the method comprises treating arthritis pain.

Optionally, the method comprises treating a focal lesion in the brain.

Optionally, the method comprises treating sleep apnea by at least one of ablation of a palate and ablation of a tongue.

Optionally, the method comprises ablating an inferior turbinate in a submucosal space to relieve chronic nasal obstruction.

Optionally, the method comprises ablating a solitary thyroid nodule to improve thyroid function.

The aforementioned and other embodiments of the present invention shall be described in greater depth in the drawings and detailed description provided below.

### BRIEF DESCRIPTION OF THE DRAWINGS

These and other features and advantages of the present invention will be further appreciated, as they become better understood by reference to the detailed description when considered in connection with the accompanying drawings, wherein:

FIG. 1 illustrates an ablation system for the ablation of animal tissue, in accordance with embodiments of the present specification;

FIG. 2 illustrates a system for use in the ablation of animal tissue, in accordance with another embodiment of the present specification;

FIG. 3 illustrates a controller for use with an ablation system, in accordance with an embodiment of the present specification;

FIG. 4A illustrates a front view of the positioning element arrangement, in accordance with some embodiments of the present specification;

FIG. 4B illustrates a side view of the positioning element arrangement, in accordance with some embodiments of the present specification;

FIG. 4C illustrates a front perspective view of the positioning element arrangement, in accordance with some embodiments of the present specification;

FIG. 4D illustrates a side view and exemplary dimensions of the positioning element arrangement 400, in accordance with some embodiments of the present specification;

FIG. 5A illustrates a front view of the positioning element arrangement, in accordance with some embodiments of the present specification;

FIG. 5B illustrates a side view of the positioning element arrangement, in accordance with some embodiments of the present specification;

FIG. 5C illustrates a front perspective view of the positioning element arrangement, in accordance with some embodiments of the present specification;

FIG. 5D illustrates a side view and exemplary dimensions of the positioning element arrangement, in accordance with some embodiments of the present specification;

FIG. 5E illustrates photographs of actual positioning element arrangements, in accordance with some other embodiments of the present specification;

FIG. 5F illustrates an enlarged view of the positioning element arrangement showing the connection, in accordance with some embodiments of the present specification;

FIG. 5G illustrates a catheter connected to a connector, in accordance with some embodiments of the present specification;

FIG. 5H illustrates an alternative embodiment of a piercing needle positioned inside inner catheter, in accordance with some embodiments of the present specification;

FIG. 6A illustrates an ablation catheter with a positioning element shaped like a wire mesh ball with one or more vapor delivery ports along the length of a catheter, in accordance with some embodiments of the present specification;

FIG. 6B illustrates an alternative spherical/elliptical embodiment of FIG. 6A as it is being manufactured, in accordance with some embodiments of the present specification;

FIG. 6C illustrates the alternative spherical/elliptical embodiment of FIG. 6B in a later step as it is being manufactured, in accordance with some embodiments of the present specification;

FIG. 6D illustrates a configuration of the distal end cap and the proximal end cap assembly, in accordance with some embodiments of the present specification;

FIG. 6E illustrates another view of the configuration of FIG. 6D showing the coaxial catheter assembly with the outer sheath of the catheter and the inner catheter;

FIG. 7A illustrates a positioning element in a compressed state, in accordance with some embodiments of the present specification;

FIG. 7B illustrates a positioning element in an expanded state, in accordance with some embodiments of the present specification;

FIG. 8A illustrates top view of a distal end of an ablation catheter having a spherical shaped distal tip segment and a cover extending over the entirety or a portion of the tip segment, in accordance with an exemplary embodiment of the present specification;

FIG. 8B illustrates a side horizontal view of the distal end of an ablation catheter having the spherical shaped distal tip segment and cover extending over the entirety or a portion of the tip segment, in accordance with an exemplary embodiment of the present specification;

FIG. 8C illustrates a side perspective view of the distal end of an ablation catheter having the spherical shaped distal tip segment and cover extending over the entirety or a portion of the tip segment, in accordance with an exemplary embodiment of the present specification;

FIG. 8D illustrates an attachment of connector of the wire mesh element to an outer catheter shaft, in accordance with some embodiments of the present specification;

FIG. 8E illustrates a displaced distal tip, which acts as a ‘bumper’ and is atraumatic to the tissue, in accordance with some embodiments of the present specification;

FIG. 9 is a flow chart illustrating an exemplary process of ablation, in accordance with some embodiments of the present specification;

FIG. 10A is a flow chart illustrating an exemplary process of treating tumor proximate a vital structure such as a blood vessel or a bowel wall, in accordance with the embodiments of the present specification;

FIG. 10B illustrates treating a tumor on a small bowel wall, in accordance with the embodiments of the present specification;

FIG. 10C illustrates treating a tumor in pancreatic cancer patients with vascular involvement, in accordance with the embodiments of the present specification;

FIG. 11A is a representation of an exemplary catheter arrangement that is used for vapor ablation of an artery that is supplying blood to a tumor, in accordance with some embodiments of the present specification;

FIG. 11B illustrates positioning of the catheter arrangement of FIG. 11A to treat a tumor that is present within liver of a patient, and is fed by hepatic artery, in accordance with some embodiments of the present specification;

FIG. 11C is a flow chart illustrating an exemplary method for TAVA of tumors such as tumor shown in FIG. 11B, using the catheter arrangement of FIG. 11A;

FIG. 11D is a flow chart illustrating another exemplary method for TAVA of tumors such as tumor shown in FIG. 11B, using the catheter arrangement of FIG. 11A;

FIG. 12A illustrates using multiple vapor ablation tools to treat pain transmitted by a nerve proximate a facet joint in a spinal motion segment of a patient, in accordance with some embodiments of the present specification;

FIG. 12B illustrates using trocar needles for administering vapor ablation using ablation tools to treat pain transmitted by nerves in different parts of a patient's body, in accordance with some embodiments of the present specification;

FIG. 12C is a flow chart illustrating an exemplary process for treating pain using RF vapor neurotomy, in accordance with the present specification;

FIG. 12D illustrates use of a vapor delivery tool to administer vapor for basivertebral nerve ablation using the RF vapor ablation procedure in accordance with the present specification;

FIG. 12E illustrates use of a vapor delivery tool with a needle to administer vapor for treating arthritis pain using the RF vapor ablation procedure in accordance with the present specification;

FIG. 12F illustrates use of the RF vapor ablation procedure to treat a tumor in the liver, in accordance with some embodiments of the present specification;

FIG. 12G illustrates MRI guided use of a vapor delivery tool to treat a focal lesion in the brain using the RF vapor ablation procedure, in accordance with some embodiments of the present specification;

FIG. 13A illustrates use of a vapor delivery tool to treat sleep apnea using the RF vapor ablation procedure, in accordance with some embodiments of the present specification;

FIG. 13B illustrates steps involved in RF vapor ablation of palate to treat sleep apnea using the ablation systems and methods in accordance with the embodiments of the present specification;

FIG. 13C is a flow chart illustrating the steps involved in RF vapor ablation of palate to treat sleep apnea using the ablation systems and methods in accordance with the embodiments of the present specification;

FIG. 14A illustrates the steps involved in RF vapor ablation of tongue to treat obstructive sleep apnea using the ablation systems and methods in accordance with the embodiments of the present specification;

FIG. 14B is a flow chart illustrating the steps involved in RF vapor ablation of tongue to treat obstructive sleep apnea using the ablation systems and methods in accordance with the embodiments of the present specification;

FIG. 15A illustrates the steps involved in RF vapor ablation of inferior turbinate in the submucosal space to relieve chronic nasal obstruction using the ablation systems and methods in accordance with the embodiments of the present specification;

FIG. 15B is a flow chart illustrating the steps involved in RF vapor ablation of inferior turbinate in the submucosal space to relieve chronic nasal obstruction using the ablation systems and methods in accordance with the embodiments of the present specification; and

FIG. 16 illustrates the steps involved in RF vapor ablation of a solitary thyroid nodule to improve thyroid function, using the ablation systems and methods in accordance with the embodiments of the present specification.

### DETAILED DESCRIPTION

“Treat,” “treatment,” and variations thereof refer to any reduction in the extent, frequency, or severity of one or more symptoms or signs associated with a condition.

“Duration” and variations thereof refer to the time course of a prescribed treatment, from initiation to conclusion, whether the treatment is concluded because the condition is resolved or the treatment is suspended for any reason. Over the duration of treatment, a plurality of treatment periods may be prescribed during which one or more prescribed stimuli are administered to the subject.

“Period” refers to the time over which a “dose” of stimulation is administered to a subject as part of the prescribed treatment plan.

The term “and/or” means one or all of the listed elements or a combination of any two or more of the listed elements.

In the description and claims of the application, each of the words “comprise” “include” and “have”, and forms thereof, are not necessarily limited to members in a list with which the words may be associated. The terms “comprises” and variations thereof do not have a limiting meaning where these terms appear in the description and claims.

Unless otherwise specified, “a,” “an,” “the,” “one or more,” and “at least one” are used interchangeably and mean one or more than one.

The term “controller” refers to an integrated hardware and software system defined by a plurality of processing elements, such as integrated circuits, application specific integrated circuits, and/or field programmable gate arrays, in data communication with memory elements, such as random access memory or read only memory where one or more processing elements are configured to execute programmatic instructions stored in one or more memory elements.

The term “vapor generation system” refers to any or all of the heater or induction-based approaches to generating steam from water described in this application.

Any and all of the needles and needle configurations disclosed in the specification with regards to a particular embodiment, such as including but not limited to, single needles, double needles, multiple needles and insulated needles, are not exclusive to that embodiment and may be used with any other of the embodiments disclosed in the specification in any of the organ systems for any condition related to the organ system.

For purposes of the present specification, ‘completely ablating’ is defined as ablating more than 55% of a surface area or a volume around an anatomical structure.

All of the methods and systems for vapor ablation may include optics to assist with direct visualization during ablation procedures.

All ablation catheters disclosed in the specification, in some embodiments, include insulation at the location of the electrode(s) to prevent ablation of tissue proximate the location of the electrode within the catheter.

For any method disclosed herein that includes discrete steps, the steps may be conducted in any feasible order. And, as appropriate, any combination of two or more steps may be conducted simultaneously.

Also herein, the recitations of numerical ranges by endpoints include all numbers subsumed within that range (e.g., 1 to 5 includes 1, 1.5, 2, 2.75, 3, 3.80, 4, 5, etc.). Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present specification. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the specification are approximations, the numerical values set forth in the specific examples are reported as precisely as possible. All numerical values, however, inherently contain a range necessarily resulting from the standard deviation found in their respective testing measurements.

The devices and methods of the present specification can be used to cause controlled focal or circumferential ablation of targeted tissue to varying depth in a manner in which complete healing with re-epithelialization can occur. Moreover, the ablation is carried out to selectively ablate cellular elements of a tissue, without significantly ablating the extra cellular matrix (ECM). Additionally, the vapor could be used to treat/ablate benign and malignant tissue growths resulting in destruction, liquefaction and absorption of the ablated tissue. The dose and manner of treatment can be adjusted based on the type of tissue and the depth of ablation needed. The ablation devices can be used for the treatment of Barrett's esophagus and esophageal dysplasia, flat colon polyps, gastrointestinal bleeding lesions, ablation of a portion of a duodenal mucosa for the treatment of various gastrointestinal (GI) disorders, and pulmonary ablation. The ablation devices can be used for treating at least one of excess weight, obesity, eating disorders, metabolic syndrome, dyslipidemia, diabetes, polycystic ovarian disease, fatty liver disease, non-alcoholic fatty liver disease, or non-alcoholic steatohepatitis disease by ablating duodenal tissue. The ablation devices can also be used for the treatment of focal or circumferential mucosal or submucosal lesions of any hollow organ or hollow body passage in the body. The hollow organ can be one of gastrointestinal tract, pancreaticobiliary tract, genitourinary tract, respiratory tract or a vascular structure such as blood vessels. The ablation devices can be used for prostate and endometrial ablation and for the treatment of any mucosal, submucosal or circumferential lesion, such as inflammatory lesions, tumors, polyps and vascular lesions. The ablation devices can also be used for the urinary bladder ablation, and for treating an over-active bladder (OAB). The ablation devices can also be used for the treatment of focal or circumferential mucosal or submucosal lesions of the genitourinary tract. Embodiments of the present specification are useful in the treatment of genitourinary structures, where the term “genitourinary” includes all genital and urinary structures, including, but not limited to, the prostate, uterus, and urinary bladder, and any conditions associated therewith, including, but not limited to, benign prostatic hyperplasia (BPH), prostate cancer, uterine fibroids, abnormal uterine bleeding (AUB), overactive bladder (OAB), strictures, and tumors. The ablation device can be placed endoscopically, radiologically, surgically or under direct visualization. In various embodiments, wireless endoscopes or single fiber endoscopes can be incorporated as a part of the device. In another embodiment, magnetic or stereotactic navigation can be used to navigate the catheter to the desired location. Radio-opaque or sonolucent material can be incorporated into the body of the catheter for radiological localization. Ferromagnetic materials can be incorporated into the catheter to help with magnetic navigation.

Ablative agents such as steam, heated gas or cryogens, such as, but not limited to, liquid nitrogen are inexpensive and readily available and are directed via the infusion port onto the tissue, held at a fixed and consistent distance, targeted for ablation. This allows for uniform distribution of the ablative agent on the targeted tissue. The flow of the ablative agent is controlled by a microprocessor according to a predetermined method based on the characteristic of the tissue to be ablated, required depth of ablation, and distance of the port from the tissue. The microprocessor uses temperature, pressure or other sensing data to control the flow of the ablative agent. In addition, one or more suction ports are provided to suction the ablation agent from the vicinity of the targeted tissue. The targeted segment can be treated by a continuous infusion of the ablative agent or via cycles of infusion and removal of the ablative agent as determined and controlled by the microprocessor.

The systems and methods of the present specification may be particularly useful for many surgical applications, such as in the ablation of various tissues, where delivering high quality (low water content) steam results in more effective treatment. It should be appreciated that, for some of the embodiments disclosed in this specification, the term ablative agent preferably refers solely to the heated vapor, or steam, and the inherent heat energy stored therein, without any augmentation from any other energy source, including a radio frequency, electrical, ultrasonic, optical, or other energy modality. Further, the steam contracts on cooling. Steam turns to water which has a lower volume as compared to a cryogen that will expand or a hot fluid used in hydrothermal ablation whose volume stays constant upon contacting the tissue. With both cryogens and hot fluids, increasing energy delivery is associated with increasing volume of the ablative agent which, in turn, requires mechanisms for removing the agent, otherwise the medical provider will run into complications, such as perforation. However, steam, on cooling, turns into water which occupies significantly less volume. Therefore, increasing energy delivery is not associated with an increase in volume of the residual ablative agent, thereby eliminating the need for continued removal.

It should be appreciated that the devices and embodiments described herein are implemented in concert with a controller that comprises a microprocessor executing control instructions. The controller can be in the form of any computing device, including desktop, laptop, and mobile device, and can communicate control signals to the ablation devices in wired or wireless form.

The present invention is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

It should be noted herein that any feature or component described in association with a specific embodiment may be used and implemented with any other embodiment unless clearly indicated otherwise.

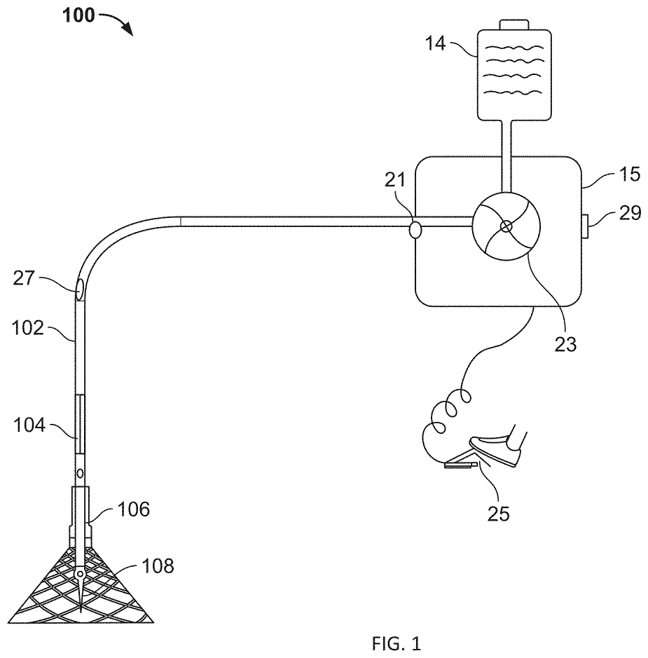

FIG. 1 illustrates an ablation system 100 suitable for use in ablating animal tissue or tissue of a patient, in accordance with some embodiments of the present specification. The ablation system 100 comprises a catheter 102 having an internal heating chamber 104, disposed within a lumen of the catheter 102 and configured to heat a fluid provided to the catheter 102 to change said fluid to a vapor for ablation therapy. In one embodiment the fluid is electrically conductive saline and is converted into electrically non-conductive or poorly conductive vapor. In one embodiment, there is at least a 25% decrease in the conductivity, preferably a 50% decrease and more preferably a 90% decrease in the conductivity, of the fluid as determined by comparing the conductivity of the fluid, such as saline, prior to passing through the heating chamber to the conductivity of the ablative agent, such as steam, after passing through the heating chamber. It should further be appreciated that, for each of the embodiments disclosed in this specification, the term ablative agent preferably refers solely to the heated vapor, or steam, and the inherent heat energy stored therein, without any augmentation from any other energy source, including a radio frequency, electrical, ultrasonic, optical, or other energy modality.

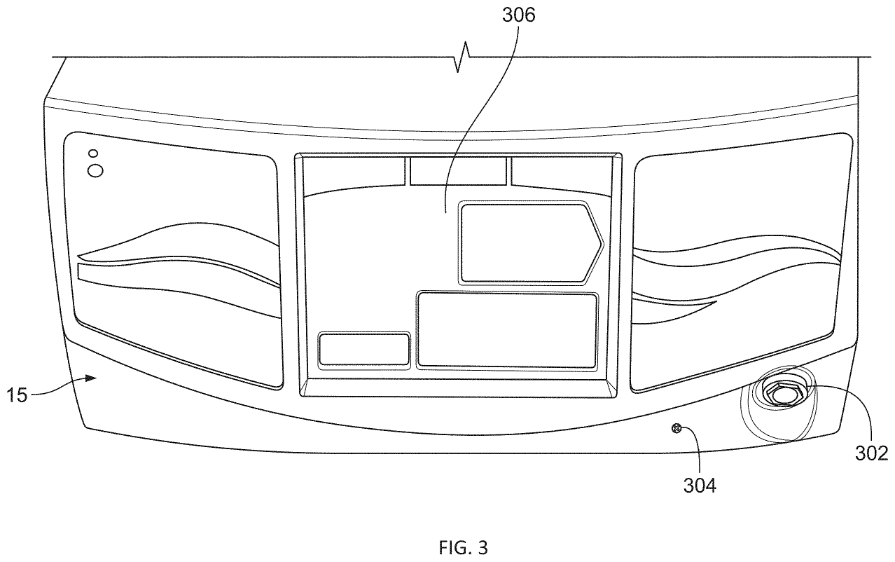

In some embodiments, the catheter 102 is made of or covered with an insulated material to prevent the escape of ablative energy from the catheter body. An opening 106 is located proximate the distal end of the catheter 102 for enabling a plurality of associated thermally conductive elements, such as one or more needles 108, to be extended and deployed or retracted through one or more openings 106. In accordance with an aspect, the needle 108 is hollow and includes at least one infusion port to allow delivery of an ablative agent, such as steam or vapor, through the needle 108 when the needle 108 is extended and deployed through the opening 106 on the elongated body of the catheter 102. In some embodiments, the infusion port is positioned along a length of the needle 108. In some embodiments, the infusion port is positioned at a distal tip of the needle 108. During use, cooling fluid such as water, air, or CO₂ is circulated through an optional port to cool the catheter 102. Vapor for ablation and cooling fluid for cooling are supplied to the catheter 102 at its proximal end. A fluid, such as saline, is stored in a reservoir, such as a saline pump 14, connected to the catheter 102. Delivery of the ablative agent is controlled by a controller 15 and treatment is controlled by a treating physician via the controller 15. An embodiment of the controller 15 is described subsequently in FIG. 3. The controller 15 includes at least one processor 23 in data communication with the saline pump 14 and a catheter connection port 21 in fluid communication with the saline pump 14. In some embodiments, at least one optional sensor monitors changes in an ablation area to guide flow of ablative agent. In some embodiments, the sensor comprises at least one of a temperature sensor or pressure sensor. In some embodiments, the catheter 102 includes a filter with micro-pores which provides back pressure to the delivered steam, thereby pressurizing the steam. The predetermined size of micro-pores in the filter determine the backpressure and hence the temperature of the steam being generated. In some embodiments, the system further comprises a foot pedal 25 in data communication with the controller 15, a switch 27 on the catheter 102, or a switch 29 on the controller 15, for controlling vapor flow. In some embodiments, the needle 108 has an attached mechanism to change its direction from being relatively parallel to the catheter 102 to being at an angle between 30°-90° to the catheter 102. In one embodiment, the aforementioned mechanism is a pull wire. In some embodiments, the opening 106 in the catheter is shaped to change the direction of the needle 108 from being relatively parallel to the catheter 102 to being at an angle between 30°-90° to the catheter 102.

In one embodiment, a user interface included with the microprocessor 15 allows a physician to define device, organ, and condition which in turn creates default settings for temperature, cycling, volume (sounds), and standard RF settings. In one embodiment, these defaults can be further modified by the physician. The user interface also includes standard displays of all key variables, along with warnings if values exceed or go below certain levels.

The ablation device also includes safety mechanisms to prevent users from being burned while manipulating the catheter, including insulation, and optionally, cool air flush, cool water flush, and alarms/tones to indicate start and stop of treatment.

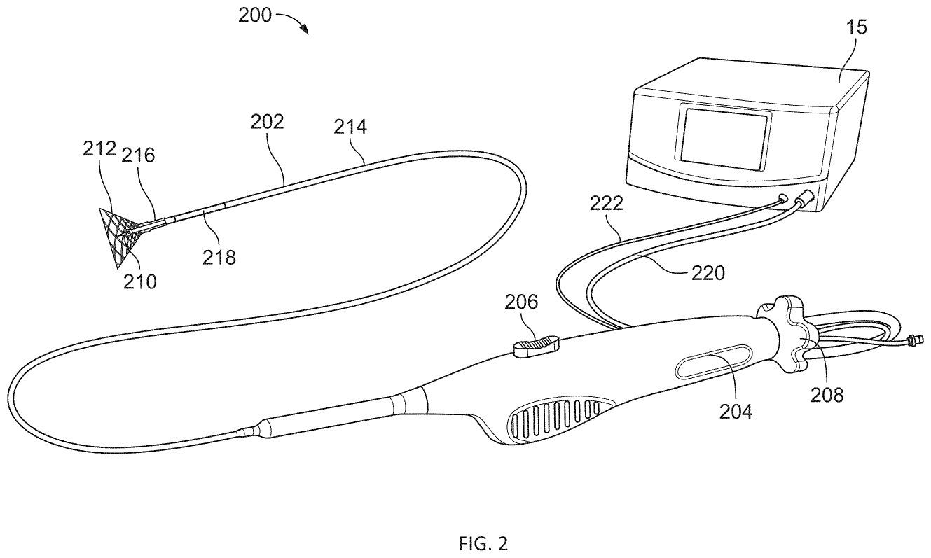

FIG. 2 illustrates a system 200 for use in the ablation of animal tissue, in accordance with another embodiment of the present specification. The system 200 comprises a catheter 202 which, in some embodiments, includes a handle 204 having actuators 206, 208 for extending at least one needle 210 or a plurality of needles from a distal end of the catheter 202 and expanding a positioning element 212 at the distal end of the catheter 202. In some embodiments, actuators 206 and 208 may be one of a knob or a slide or any other type of switch or button to enable extending of the needle 210. Delivery of vapor via the catheter 202 is controlled by a controller 15. In embodiments, the catheter 202 comprises an outer sheath 214 and an inner catheter 216. The needle 210 extends from the inner catheter 216 at the distal end of the sheath 214 or, in some embodiments, through an opening proximate the distal end of the sheath 214. In embodiments, the positioning element 212 is expandable, positioned at the distal end of the inner catheter 216, and may be compressed within the outer sheath 214 for delivery. In some embodiments, actuator 208 comprises a knob which is turned by a first extent, for example, by a quarter turn, to pull back the outer sheath 214. As the outer sheath 214 retracts, the positioning element 212 is revealed. In embodiments, the positioning element 212 is configured in the shape of a hood. The needle 210 pierces a tissue to position the needle 210 within a target tissue to be ablated while the positioning element 212 captures any vapor escaping along the needle tract due to backpressure. Optional cooling mechanisms can be incorporated into the positioning element 212 to cool the surface of the tissue while ablating inside the targeted tissue. In embodiments, actuator/knob 208 is turned by a second extend, for example, by a second quarter turn, to pull back the outer sheath 214 further to deploy the needle 210. In some embodiments, the number of needles that is deployed is two or more than two.

Referring again to FIG. 2, in some embodiments, the catheter 202 includes a port for the delivery of fluid, for example cooling fluid, during ablation. In some embodiments, the port is also configured to provide for fluid collection, provide vacuum, and provide CO₂ for an integrity test. In some embodiments, the port is positioned on the handle 204. In some embodiments, at least one electrode 218 is positioned at a distal end of the catheter 202 proximal to the needle 210. The electrode 218 is configured to receive electrical current, supplied by a connecting wire 220 extending from the controller 15 to the catheter 202, to heat and convert a fluid, such as saline supplied via tubing 222 extending from the controller 15 to the catheter 202. Heated fluid or saline is converted to vapor or steam to be delivered by needle 210 for ablation.

FIG. 3 illustrates a controller 15 for use with an ablation system, in accordance with an embodiment of the present specification. Controller 15 controls the delivery of the ablative agent to the ablation system (100, 200 of FIGS. 1 and 2, respectively). The controller 15 therefore provides a control interface to a physician for controlling the ablation treatment. An input port 302 on the controller 15 provides a port to connect the controller 15 to the catheter and provide electrical signal to the catheter. A fluid port 304 on the controller 15 provides a port for connecting a supply to fluid such as saline through a tubing to the catheter. In embodiments, a graphical user interface (GUI) 306 on the controller 15 shows the settings for operating the ablation system, which may be in use and/or modified by the physician during use. In some embodiments, the GUI 306 is a touchscreen allowing for control of the system 15 by a user. Sensors at the distal end of the catheter provide temperature and pressure observations, which are used by the controller 15 to regulate the delivery of the ablative agent.

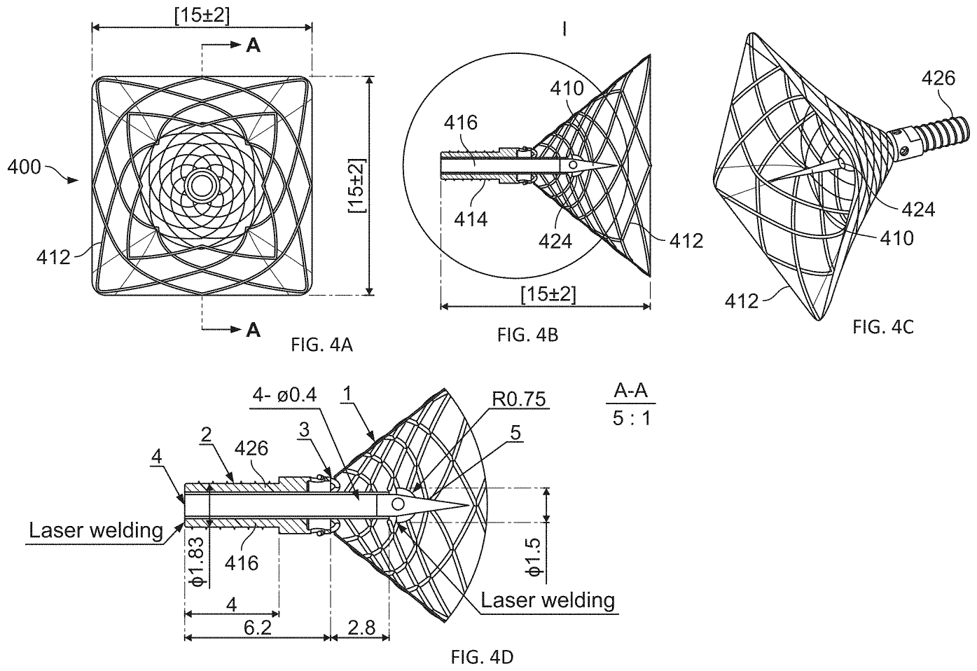

FIGS. 4A to 4D illustrate different views of a positioning element arrangement 400, in accordance with some embodiments of the present specification. FIG. 4A illustrates a front view of the positioning element arrangement 400, in accordance with some embodiments of the present specification. FIG. 4B illustrates a side view of the positioning element arrangement 400, in accordance with some embodiments of the present specification. FIG. 4C illustrates a front perspective view of the positioning element arrangement 400, in accordance with some embodiments of the present specification. FIG. 4D illustrates a side view and exemplary dimensions of the positioning element arrangement 400, in accordance with some embodiments of the present specification. The positioning element arrangement 400 comprises a positioning element 412, such as a hood-shaped positioning element. Element 412 corresponds to the positioning element 212 of FIG. 2, and is positioned at a distal end of the catheter 202. Referring simultaneously to FIGS. 4A to 4D, the positioning element 412 is illustrated in its expanded state, after a needle 410 has extended from an inner catheter 416 at a distal end of an outer sheath or an opening at a distal end of an outer sheath 414. In embodiments, the needle 410 is a thermocouple needle configured to monitor temperature changes at the site of target tissue for ablation. The needle 410 is a piercing needle with one or more ports that may be located along a length of the needle. The figure illustrates at least one port 424, which comprises an opening that provides a path for vapor to exit for ablation. In embodiments, the positioning element 412 is expandable, positioned at the distal end of the inner catheter 416, and may be compressed before use within the outer sheath. The figures illustrate an embodiment of element 412 that has a pyramid-shape, with the base of the square pyramid opening at a distal side of the element 412. In some embodiments, the square has a side of approximately 13 to 17 millimeters (mm). In some embodiments, the positioning element 412 has a wire mesh structure with or without a covering membrane. In some embodiments, the element 412 is made of a bioresorbable material and resorbs after a predetermined time. In some embodiments, the element 412 has a constraining and/or removing mechanism attached to it for removal at a later date. In some embodiments, the constraining and or removing mechanism is a PTFE, ePTFE or silk suture. In some embodiments, the element 412 is made of ECM to help proper healing of the tissue post-ablation. In some embodiments, the elements 412 are made from Nitinol wire meshes. The wires may have a diameter in a range of 0.16 to 0.18 mm. In some embodiments, for the positioning element 412, the wire mesh is coated with silicone but not the areas between wires in the mesh, therefore allowing steam to escape/vent from these spaces between the wires.

In some embodiments, the positioning element arrangement 400 can be removably attached to an opening at the distal end of the outer sheath, with a connector 426. The connector 426 may comprise helical guide grooves on a shaft adapted for receiving inside a distal opening of the outer sheath 414 upon rotation of the shaft while moving towards a direction of outer sheath 414 along a longitudinal axis of shaft and outer sheath 414. In some embodiments, a proximal length of the shaft, including a side that faces the outer sheath 414, has a diameter of 1.83 mm, and extends for a length of 4 mm. The helical grooves are configured on an outer surface of the proximal length of the shaft. A distal length of the shaft may have a diameter slightly larger (approximately 2.3 mm) than the proximal length of the shaft. A total length of the shaft (proximal and distal) is approximately 6.2 mm. The element 412 is attached to the distal end of the distal length on shaft. In some embodiments, the inner catheter 416 extends for another 2.8 mm length from the distal end of the distal length of shaft, with the needle 410 attached to the distal end of the inner catheter 416, such that the needle 410 remains within the hood of element 412 after expanding the element 412 for delivery of ablation. A base of the needle, which is the side of the needle 410 that is opposite to the piercing side of the needle 410, is attached to the inner catheter 416 by means such as and not limited to laser welding. The base of the needle 410 corresponds to a diameter of the inner catheter 416, which is approximately 1.5 mm. In some embodiments, cooling mechanisms are incorporated into the hood to cool the surface of the tissue while ablating inside the targeted tissue. The length of the needle 410 extending from the distal end of the shaft of connector 426 to its piercing tip is approximately 5 mm.

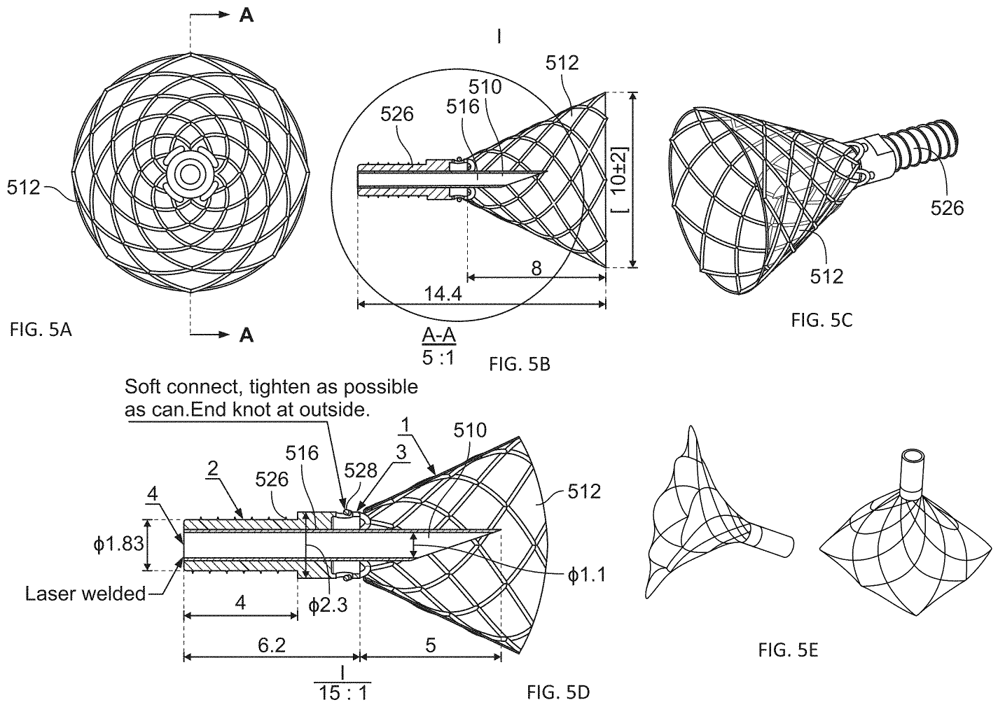

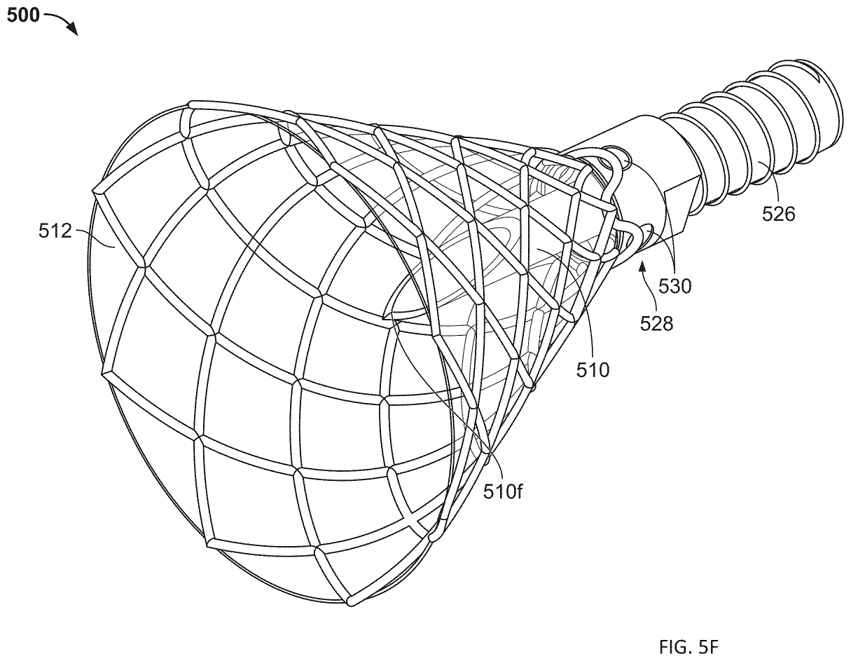

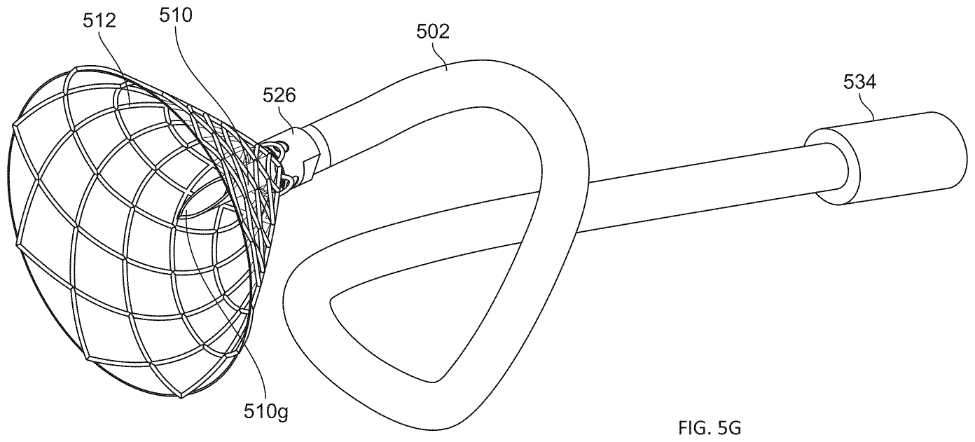

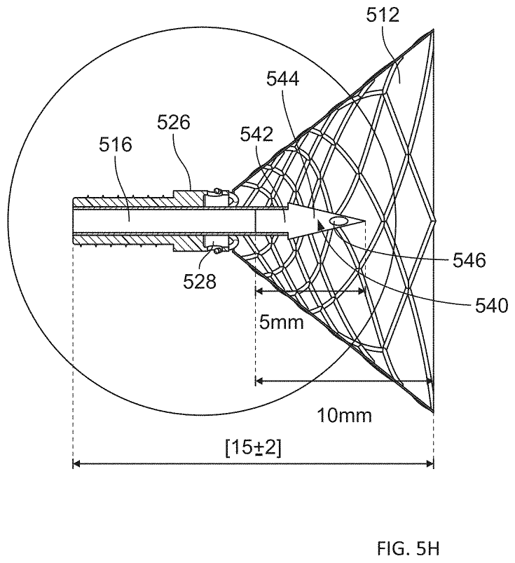

FIGS. 5A to 5D illustrate a positioning element arrangement 500, in accordance with some other embodiments of the present specification. FIG. 5A illustrates a front view of the positioning element arrangement 500, in accordance with some embodiments of the present specification. FIG. 5B illustrates a side view of the positioning element arrangement 500, in accordance with some embodiments of the present specification. FIG. 5C illustrates a front perspective view of the positioning element arrangement 500, in accordance with some embodiments of the present specification. FIG. 5D illustrates a side view and exemplary dimensions of the positioning element arrangement 500, in accordance with some embodiments of the present specification. Elements of FIGS. 5A to 5D can be described similar to elements of FIGS. 4A to 4D, except that the hood of a positioning element 512 is shaped in the form of a circular cone with a diameter in a range of 8 to 12 mm. FIG. 5E illustrates photographs of actual positioning element arrangements 500, in accordance with some other embodiments of the present specification. Length of the cone of element 512 is approximately 8 mm. FIGS. 5A to 5D also illustrate a connection 528 between a connector 526 and the element 512. FIG. 5F illustrates an enlarged view of the positioning element arrangement 500 showing the connection 528, in accordance with some embodiments of the present specification. Connection 528 is formed by tying a wire that passes through a series of equally-distant holes around the circumference of the distal end of the distal shaft of the connector 526. The wire is entwined with the positioning element 512 as tightly as possible. The wire may terminate with a knot outside the positioning element 512. FIG. 5G illustrates a catheter 502 connected to the connector 526, in accordance with some embodiments of the present specification. The connector 526 is connected at the distal end of the catheter 502. At the proximal end, a port 534 may be provided for input of fluids for ablation.

In some embodiments, a piercing needle 510 is positioned inside an inner catheter 516. In some embodiments, the inner catheter 516 which includes a hollow shaft through which an ablative agent can travel, comprises a puncturing tip 510f/510g at its distal end that is configured to deliver an ablative agent to the tissue. In embodiments, the needle 510 is a thermocouple needle configured to monitor the temperature changes at the site of target tissue. The ablative agent is delivered when the puncturing tip 510f/510g is extended and deployed through the distal end of the connector 526.

FIG. 5H illustrates an alternative embodiment of a piercing needle 540 positioned inside inner catheter 516, in accordance with some embodiments of the present specification. Needle 540 comprises a hollow tubular portion 542 at a proximal side that connects the needle 540 to the inner catheter 516. A length of the needle 540 from the distal tip of inner catheter 516 to the needle's 540 distal tip is approximately 5 mm. A distal portion of the needle 540 includes a pointed circular conical structure 544 that is configured to pierce a target tissue. A steam port 546 is provided in the form of a hole in the structure 544 that enables steam to escape from within the needle to ablate the target tissue.

Referring simultaneously to FIGS. 5A to 5H, positioning element 512 comprises a circular hood. In some embodiments, diameter of the hood of positioning element 512 extends in a range of 10 to 15 mm. A linear distance extending from the proximal edge of the connector 526 to the circle formed by the distal edge of the circular hood of positioning element 512 is in a range of approximately 13 to 17 mm. A linear distance of the circle formed by the distal edge of the circular hood of positioning element 512 from the distal tip of inner catheter 516 is approximately 10 mm.

Now referring to FIGS. 4A to 4D and 5A to 5H, in embodiments, the needle 410/510/540 is configured to pierce a surface of the target tissue while the hood of positioning element 412/512 rests on the surface of the target tissue, surrounding the needle 410/510/540. The vapor delivered through the steam port of the needle 410/510/540 is injected within the tissue to an area where the needle 410/510/540 is pierced. The hood of the positioning element 412/512 applies the vapor to the surface of the tissue. In embodiments, tip of needle 410/510/540 is extended to a desired length from the hood to control the depth of ablation. The desired depth of ablation may depend on the size of a lesion that needs to be ablated.

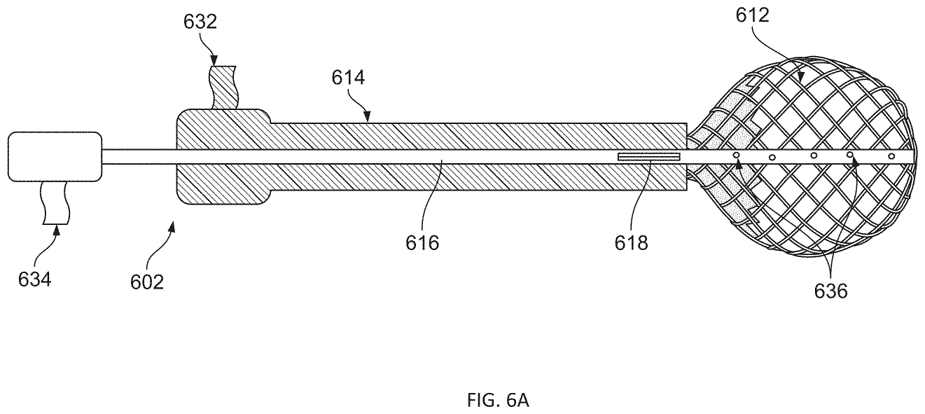

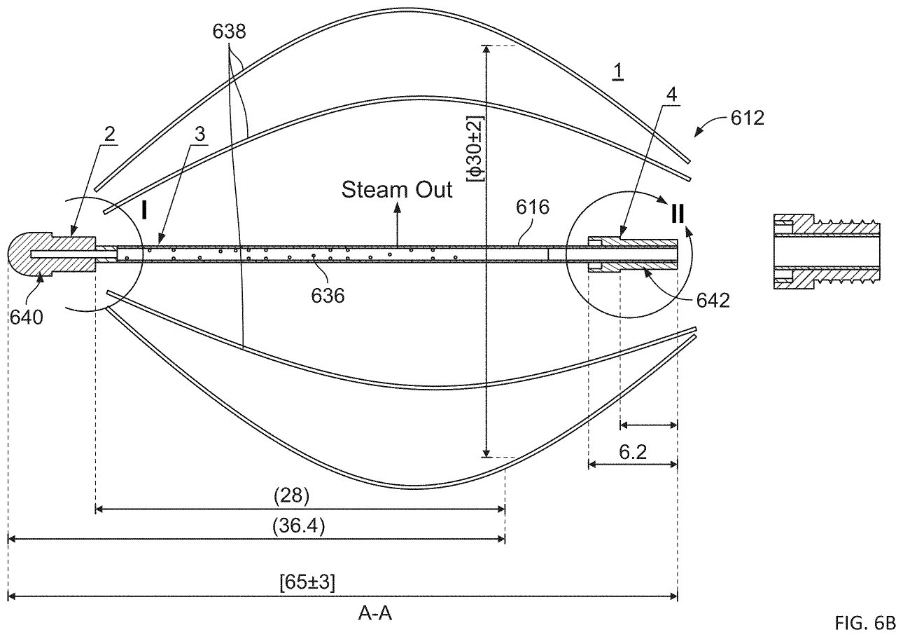

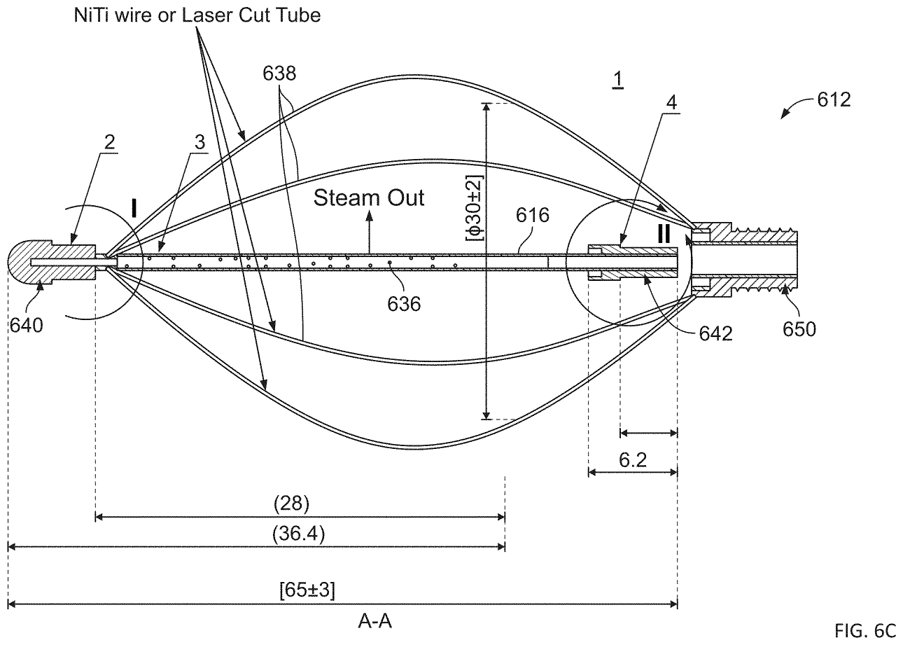

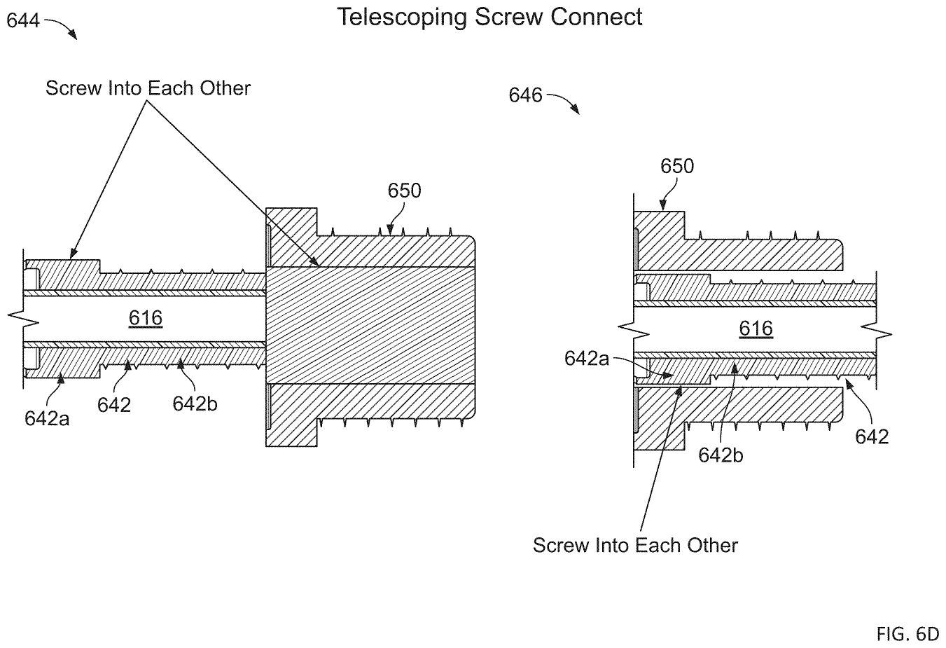

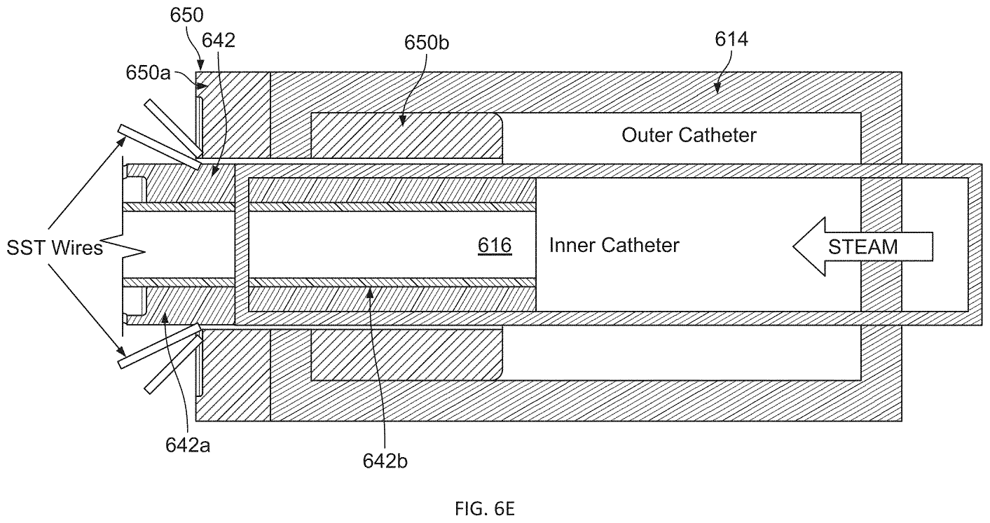

FIG. 6A illustrates an ablation catheter with a positioning element 612 shaped like a wire mesh ball with one or more vapor delivery ports 636 along the length of a catheter 602, in accordance with some embodiments of the present specification. Fluids for ablation are input from a port 634 into the catheter 602. The fluid is converted to vapor by an internal heating chamber 618 in an inner shaft 616 of the catheter 602. The inner shaft 616 is positioned within an outer sheath 614 of the catheter 602. In some embodiments, the internal heating chamber 618 comprises an RF electrode or an array of electrodes that are separated from thermally conductive element by a segment of the catheter 602 which is electrically non-conductive. In some embodiments, the catheter 602 is made of or covered with an insulated material to prevent the escape of ablative energy from the catheter body. Ablative agents such as steam, heated gas or cryogens, such as, but not limited to, liquid nitrogen are inexpensive and readily available and are directed via the infusion port onto the tissue, held at a fixed and consistent distance, targeted for ablation. This allows for uniform distribution of the ablative agent on the targeted tissue. The flow of the ablative agent is controlled by a microprocessor according to a predetermined method based on the characteristic of the tissue to be ablated, required depth of ablation, and distance of the port from the tissue. The microprocessor uses temperature, pressure or other sensing data to control the flow of the ablative agent. In addition, one or more suction ports 632 are provided to suction the ablation agent from the vicinity of the targeted tissue. The targeted segment can be treated by a continuous infusion of the ablative agent or via cycles of infusion and removal of the ablative agent as determined and controlled by the microprocessor.

In embodiments, a distal length of the inner catheter 616 includes one or more ports 636 for delivery of vapor for ablation. The catheter 602 includes the positioning element 612 that encompasses the vapor ablation ports 636. Element 612 is an expandable ball-shaped wire mesh positioning element which may or may not include a covering membrane. The wire mesh positioning element is constrained by the outer catheter 614 to be placed proximate a targeted tissue. On deployment, the positioning element 612 creates an air-filled space into which the vapor is delivered through the vapor delivery ports 636 to create tissue ablation. Optional suction is provided by suction port 632 to suction fluid out of the air-filled space during a session of vapor thermal ablation therapy.

FIG. 6B illustrates an alternative spherical/elliptical embodiment of FIG. 6A as it is being manufactured, in accordance with some embodiments of the present specification. FIG. 6C illustrates the alternative spherical/elliptical embodiment of FIG. 6B in a later step as it is being manufactured, in accordance with some embodiments of the present specification. FIG. 6B illustrates a set of components prior to assembly, at least a portion of which are used to configure the wire mesh structure 638. FIG. 6C illustrates an assembled configuration of the expandable wire mesh structure 638. Referring simultaneously to FIGS. 6B and 6C, an expandable wire mesh structure 638 is used to form the positioning element 612. In some embodiments, the expandable wire mesh structure 638 comprises laser cut nitinol tubes. The internal catheter 616 in which steam is generated and exit through vapor ablation ports 636, has two end caps—a distal end cap 640 and a proximal end cap 642. When not deployed, proximal wall of distal end cap 640 is near or adjacent to distal side of proximal end cap 642. During deployment, inner catheter 616 is moved coaxially and telescopically outside outer catheter 614 such that distal end cap 640 moves forward with the movement of inner catheter 616, while proximal end cap 642 moves only slightly but remains attached to distal end of outer catheter 614. Inner catheter 616 is coaxially positioned within proximal end cap 642 and is configured to move longitudinally along its central axis within a hollow cylindrical space of proximal end cap 642. A distal end of inner catheter 616 is attached to a proximal side of distal end cap 640. Distal end of end cap 640 acts as a ‘bumper’ and is atraumatic to the tissue. A proximal side of distal end cap 640 is attached to distal ends of wire mesh structure 638 including the nitinol tubes.

Further, proximal end cap 642 is telescopically aligned within outer catheter 614. FIG. 6E illustrates inner catheter 616 positioned within proximal end cap 642 that is further positioned coaxially within an outer catheter 614. Proximal end cap 642 includes two portions—a distal portion 642a that has a larger diameter than a proximal portion 642b, such that distal portion 642a is continually attached to proximal portion 642b. In embodiments, grooves on the outer surface of distal portion 642a are configured to screw into an inner portion of another cap 650 that is attached to a distal end of outer catheter 614. Cap 650 is also cylindrical and coaxial with cap 642. Cap 650 is configured with two continually attached portions—a distal portion 650a, and a proximal portion 650b with a diameter less than the distal portion 650a. Outer catheter 614 is attached with cap 650 such that proximal portion 650b lies inside outer catheter 614 and distal portion 650a lies outside outer catheter 614. Further, proximal ends of wire mesh structure 638 including nitinol tubes are attached to the distal end of distal portion 650a of cap 650.

Each nitinol tube of the expandable wire mesh structure 638 is longer than the extent of the catheter 616 length around which those tubes are placed. A first distal end of the tubes is connected to a proximal side of the distal end cap 640, and a second proximal end is connected to the distal side of cap 650 (distal side of distal portion 650a). Proximal portion 642b of proximal end cap 642 is configured to move laterally along the length of the catheter 616 and telescopically and longitudinally in and out of the cap 650. In embodiments, distal portion 642a of proximal end cap 642 screws into cap 650, and specifically adjacent to internal surface of distal portion 650a of cap 650. That way, by manipulating the position of the most distal end cap 640, the wires of wire mesh structure 638 are caused to extend outward (if end cap 640 is moved proximally) or to lay flat, parallel to the internal catheter 616 (if end cap 640 is moved distally).

A distance between any two most distant wires of the expandable wire mesh structure 638 is in a range of 28 to 32 mm, when measured at approximately 28 mm from where the expandable wire mesh structure 638 meets the end cap 640. The distance from the distal tip of end cap 640 to the point where the length is measured in a range of 28 to 32 mm is approximately 36.4 mm. Therefore, in some embodiments, the end cap 640 has a length of approximately 8.4 mm. A length of the proximal end cap 642 may be approximately 6.2 mm.

FIG. 6D illustrates a configuration of the proximal end cap 642 and the cap 650, in accordance with some embodiments of the present specification. The proximal end cap 642 may be moved telescopically in and out of the 650. Rotational and longitudinal movement of either or both end caps 642 and 650 enable grooves on an outer surface of the proximal end cap 642 to screw into the inner surface of the cylindrical form of cap 650. As the inner catheter 616 moves distally (with proximal end cap 642), as shown in view 644, to the length of proximal end cap 642. A further pushing out of inner catheter 616 results in distal end cap 640 moving forward as the nitinol tubes of expandable wire mesh structure 638 straighten out. As the inner catheter 616 moves proximally (pulled back), as shown in view 646, the tubes bend outward.

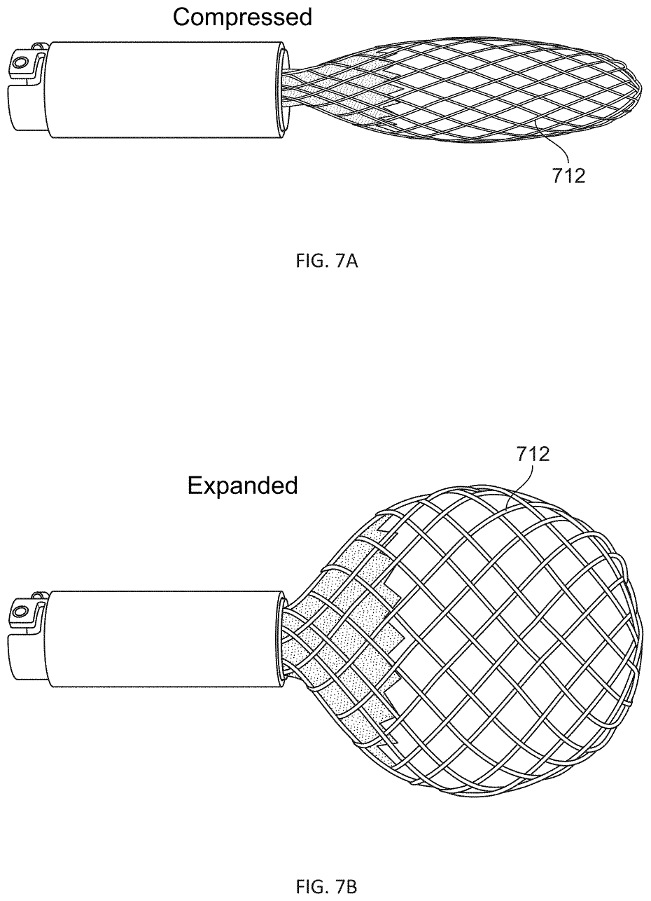

FIG. 7A illustrates a photograph of an actual positioning element 712 in a compressed state, in accordance with some embodiments of the present specification. FIG. 7B illustrates a photograph of the positioning element 712 in an expanded state, in accordance with some embodiments of the present specification. The element 712 remains in a compressed state of FIG. 7A for delivery through a lumen of an endoscope. The element 712 expands (FIG. 7B) upon deployment for treatment. In some embodiments, the positioning element 712 is expandable, positioned at the distal end of the inner catheter (616 of FIG. 6), and may be compressed within the outer sheath (614 of FIG. 6) for delivery. In some embodiments, an actuator (206, 208 of FIG. 2) comprises a knob which is turned by a first extent, for example, by a quarter turn, to pull back the outer sheath. As the outer sheath retracts, the positioning element 712 is revealed.

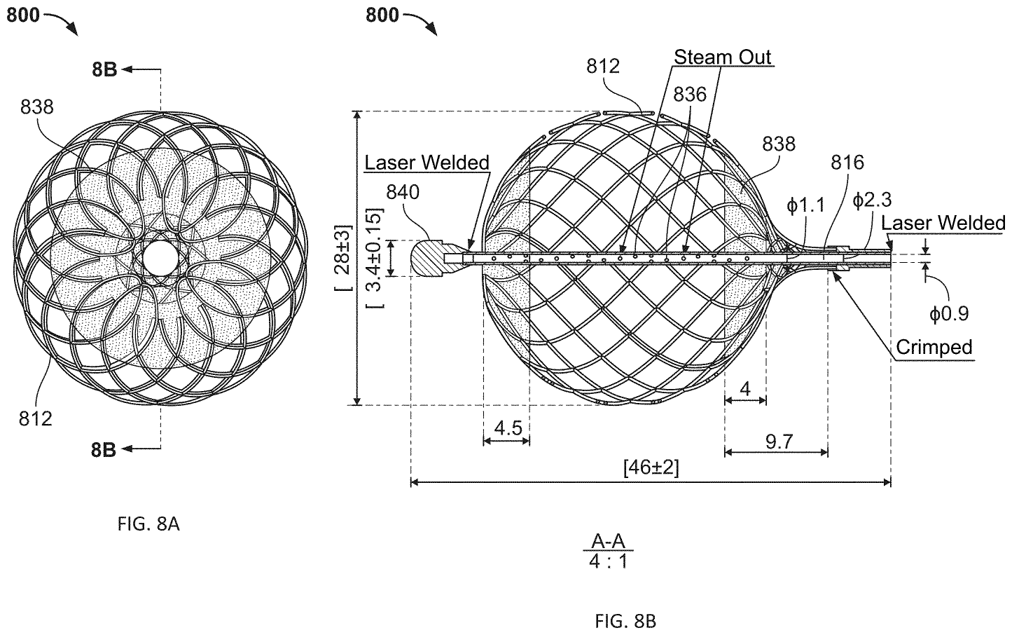

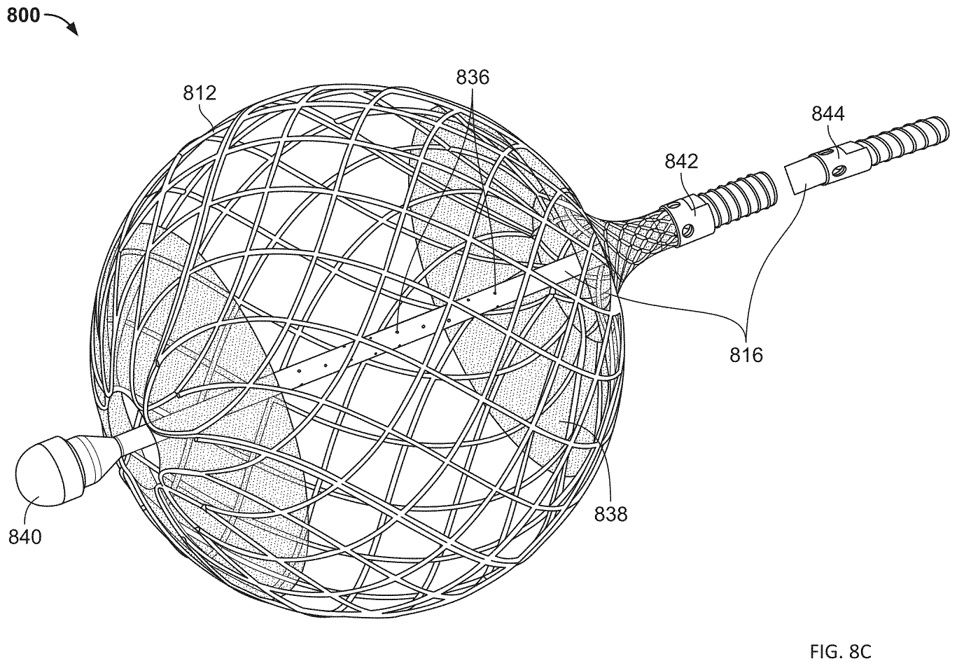

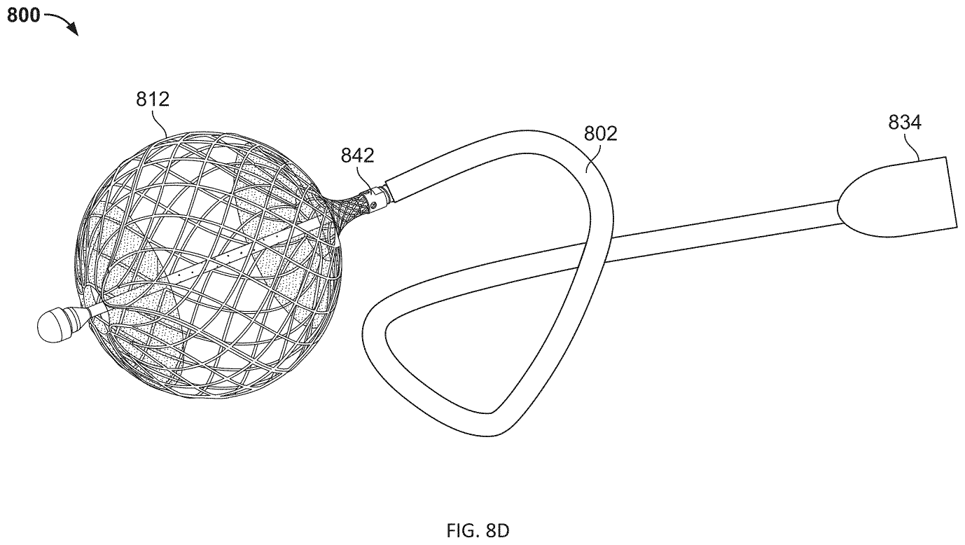

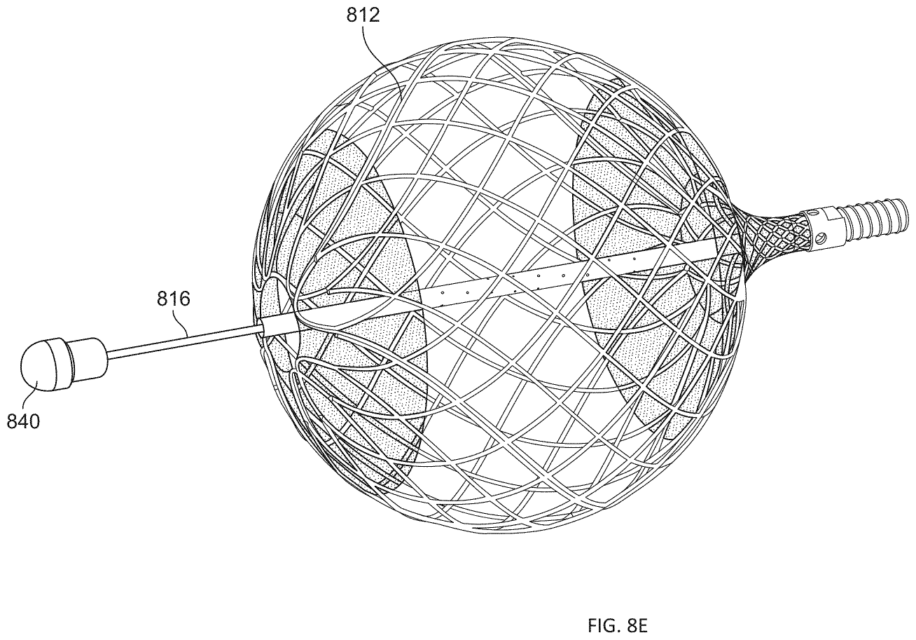

FIG. 8A illustrates top view of a distal end 800 of an ablation catheter having a spherical or elliptical shaped distal tip segment 812 and a cover 838 extending over the entirety or a portion of the tip segment 812, in accordance with an exemplary embodiment of the present specification. FIG. 8B illustrates a side horizontal view of the distal end 800 of an ablation catheter having the spherical shaped distal tip segment 812 and cover 838 extending over the entirety or a portion of the tip segment 812, in accordance with an exemplary embodiment of the present specification. FIG. 8C illustrates a side perspective view of the distal end 800 of an ablation catheter having the spherical shaped distal tip segment 812 and cover 838 extending over the entirety or a portion of the tip segment 812, in accordance with an exemplary embodiment of the present specification. Embodiments of FIGS. 8A, 8B, and 8C, may be used in catheter devices for tissue ablation. Referring simultaneously to FIGS. 8A, 8B, and 8C, a distal tip 840 is attached to a distal end of a catheter shaft and extends into the tip segment 812, which perform the function of a positioning element. The distal tip 840 may have incorporated therein or coupled thereto one or more sensors, including temperature, pressure, moisture, or other physiological sensors. The distal tip 840 is an extension of the catheter shaft 816 and is configured to have a smooth rounded tip at its most distal end. In some embodiments, the distal tip 840 is soft and is configured to have a semi-spherical shape. A portion of the distal length of shaft 816 has at least one or a plurality of openings 836 to provide an exit for vapor during ablation. In some embodiments, the openings 836 are circular, slotted, semi-circular, or of any other shape. In some embodiments, 1 to 1000 openings 836 are distributed over a length of 3 to 7 cm across the length and surface of the distal length of shaft 816, where each opening has a length or a diameter in a range of 0.1 to 1 mm. In embodiments, the distal length of shaft 816 is encompassed within the spherical element 812. The element 812 remains in a compressed state for delivery through a lumen of an endoscope. The element 812 expands into a spherical shape upon deployment for treatment. A tip of each wire mesh tip segment 812 is free floating and they are attached to the respective catheter at the proximal neck of the distal length of catheter shaft 816. In some embodiments, the wire mesh tip segment 812 is attached to a connector 842 at the proximal side. Connector 842 comprises a distal portion that provides an attachment mechanism to attach the wire mesh tip segment 812, and a proximal portion that is in the form of a tube with circular grooves on the outer surface of the tube, which are used to attach the connector 842 within an outer shaft of the catheter. The tube of connector 842 is internally hollow, so as to enable receiving of an inner catheter shaft 816. In some embodiments, the wire mesh is crimped to attach the element 812 to the catheter shaft 816 at its proximal side. At a distal side of the element 812, the wire mesh meets proximal to distal tip 840, which acts as a ‘bumper’ and is atraumatic to the tissue. Segment 812 is configured from a wire mesh so that there is sufficient space between the wires of the mesh for steam to exit. The cover 838 is provided to partially cover the openings through the wire mesh on a proximal (bottom) and distal sides of the spherical segment 812 to prevent steam from flowing in these directions. In some embodiments, cover 838 is silicone.

FIG. 8D illustrates an attachment of connector 842 of the wire mesh element 812 to an outer catheter shaft 802, in accordance with some embodiments of the present specification. The inner catheter shaft 816 emerges from within the outer catheter shaft 802, through the connector 840, to within element 812. At the proximal end of outer shaft 802, a port 834 may be provided for input of fluids for ablation.

FIG. 8E illustrates a displaced distal tip 840, which acts as a ‘bumper’ and is atraumatic to the tissue. A position of the distal tip 840 is adjustable relative to its distance from the wire mesh element 812. The inner catheter shaft 816 is pushed forward to emerge further out from within the outer catheter shaft 802 (not shown), thereby carrying forth the distal tip forward. The wire mesh tip element 812 remains attached to the distal end of the outer catheter shaft 802, and does not move with the movement of the inner catheter shaft 816. The openings 836 that provide an exit for vapor during ablation are therefore made available outside the wire mesh tip element 812, and may additionally be available with the inner catheter shaft 816 that is still positioned within the element 812. Position of the element 812 can thus be adjusted at a location where it is needed while the ablation is performed from within or outside the element 812.

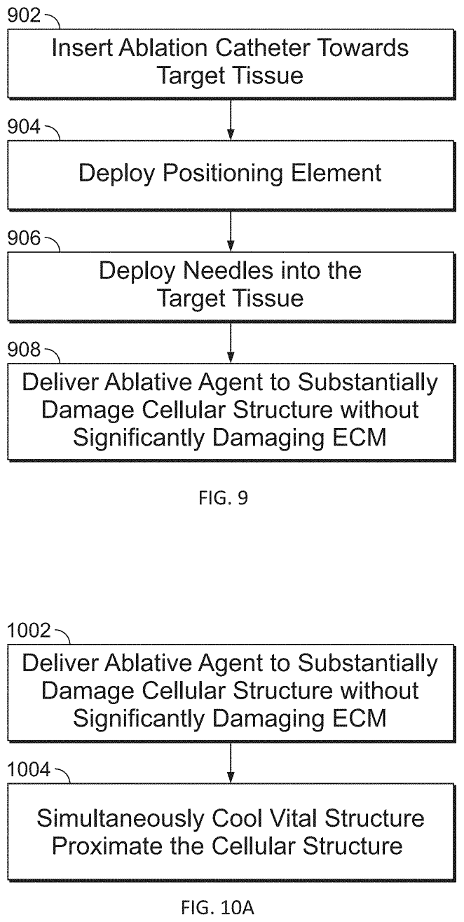

Embodiments of the present specification selectively ablate cellular elements of animal tissue without significantly ablating the ECM, thereby allowing for the tissue to heal adequately after an ablation procedure without resulting in a complication. The complications may include bleeding or stricture formation. Selective ablation is achieved by controlling the parameters of an ablative agent. In embodiments, the systems and methods of ablation of the present specification achieve ablation of greater than 50% cellular structure and less than 50% of the ECM in the target tissue. FIG. 9 is a flow chart illustrating an exemplary process of ablation, in accordance with some embodiments of the present specification. At step 902, an ablation system is provided with a catheter that is in fluid communication with a pump. The catheter transports fluid supplied by the pump. One or more thermally conductive elements such as electrodes, near a distal end of the catheter are configured to heat the fluid that is transported through a lumen (inner catheter shaft) and convert it to vapor. The vapor exits through one or more openings in the distal end of the catheter. The openings are located at either a distal length of the catheter or in a needle attached to a distal tip of the catheter. Exemplary layout of the ablation system is described in context of FIGS. 1 and 2, and may be referred here for details. The distal end of the catheter is extended towards the tissue surface (target tissue) for ablation. At step 904, a positioning element at the distal end of the catheter is expanded to activate the catheter for ablation. Embodiments of the positioning element and distal end of catheter are described in context of FIGS. 4A to 4D, 5A to 5F, 6, 7A, 7B, and 8A to 8C. At step 906, optionally a thermocouple needle is deployed from the catheter into the target tissue. The temperature measured by the thermocouple needle is used to ablate target tissue at a specific temperature and for a specific time period to achieve a differential effect on normal cellular structure, ECM, and tumorous cells (where applicable). At step 908, an ablative agent (vapor) is delivered at a temperature range of 99° C. to 110° C. through the one or more openings at the distal end of the catheter to ablate the target tissue.

In embodiments, a quality of the vapor is maintained at a level greater than 25%. A higher quality of vapor has low water content, which results in a more effective treatment. The ablation is controlled by a controller connected to the ablation system, so that the vapor results predominantly in damage or death of the cellular component in the target tissue without significantly damaging the ECM. This is possible since ECM is more resistant to thermal injury than cellular structure. Cells are damaged instantly at approximately 60° C., whereas ECM material like collagen begin to denature at temperatures above 70° C. to 75° C., after an exposure of at least a few seconds. Therefore, the controller optimizes dosimetry for different applications so that a temperature of approximately 60° C. is achieved at a deepest point in the target tissue, with very low exposure times. In some exemplary embodiments, esophagus tissue is exposed for approximately 3 to 5 seconds; duodenum tissue is exposed for approximately 3 to 5 seconds, prostate tissue is exposed for approximately 10 seconds; endometrium tissue is exposed for approximately 30 to 60 seconds; and heart tissue is also exposed for approximately 30 to 60 seconds. In embodiments, greater than 50% of the cellular component undergoes irreversible damage by the ablative agent, and less than 50% of the ECM is similarly affected. In embodiments, the controller is programmed to perform the ablation so that pressure in the target tissue is maintained at a level below 5 atm.

Embodiments of the present specification can be used for cleaning tumor margins after resection. Embodiments described in context of FIGS. 6 to 8E may be used to treat tumor margins. The wire mesh structures of the stated embodiments is deployed in the resected tumor bed and vapor is sprayed through a plurality of holes or ports on the inner catheter shaft to ablate the residual tumor in the tumor bed. Current practice of hyperthermia can be combined with embodiments of the present specification, to deliver heat and irreversibly damaged blood vessels of tumor cells without substantially damaging normal cells. Conventional local hyperthermia is usually carried out for 60 to 90 minutes at a target temperature of 39.5° C. to 43° C. Surgical guides, such as Breast Cancer Locators (BCL™) provide information regarding tumor size, shape, and margin boundary to assist surgeons in the excision of cancer and preserve normal breast tissue. Lateral marking needles in such guides can be replaced with thermocouple needles and the vapor passed through the central needle can be used to ablate the tumor at temperatures less than 60° C., or ablate the margins after surgery, in accordance with the systems and methods of the present specification. Temperature signal from the thermocouple needles can be used to guide the therapy and also the placement/repositioning of the central vapor catheter. Therefore, tumorous cells are damaged while avoiding damage to normal cells through a vascular mechanism.

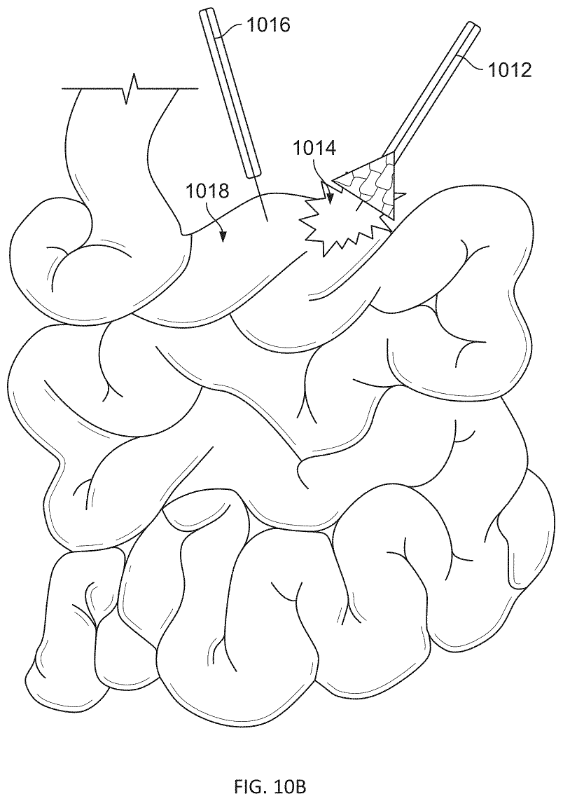

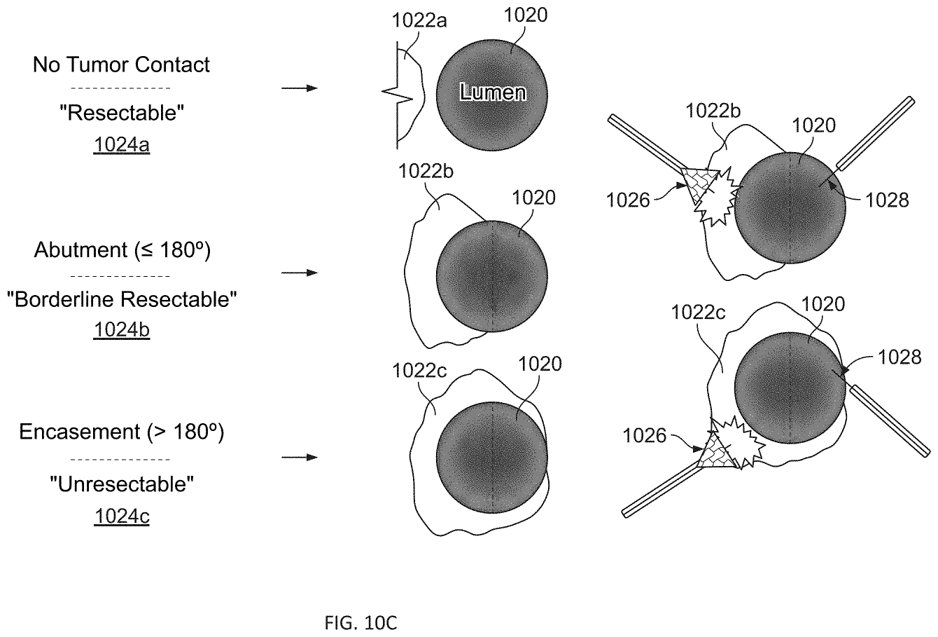

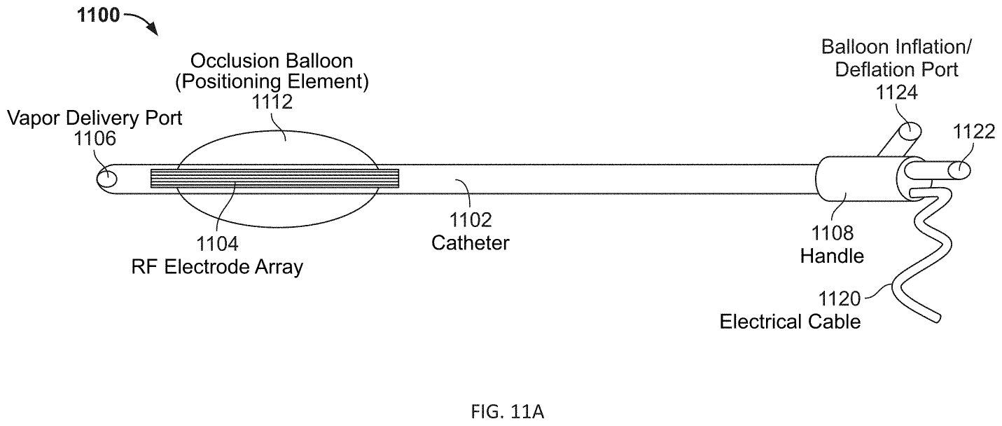

Embodiments of the present specification provide systems and methods for ablating a cellular structure, such as a tumor, proximate a vital structure. FIG. 10A is a flow chart illustrating an exemplary process of treating tumor proximate a vital structure such as a blood vessel or a bowel wall, in accordance with the embodiments of the present specification. At step 1002, vapor is injected into the cellular structure of the tumor to ablate a substantial portion of the cellular structure without ablating a substantial portion of the ECM and the vital structure, as described with reference to FIG. 9. At step 1004, the vital structure that is proximate the cellular structure is simultaneously cooled. The structure is cooled by injecting a coolant such as cold saline, at a temperature less than 37° C., into the vital structure, while simultaneously delivering vapor to the cellular structure. In an example, cold saline is injected into a bowel lumen while ablating a tumor involving an adjacent bowel wall. FIG. 10B illustrates treating a tumor on a small bowel wall. A catheter arrangement 1012 ablates the tumor 1014 by delivering vapor into or on the surface of the cellular structure of the tumor.

Another catheter arrangement 1016 simultaneously injects a coolant into the small bowel lumen 1018 using an injection needle, proximate to the tumor 1014. FIG. 10C illustrates treating a tumor in pancreatic cancer patients with vascular involvement. The illustration describes a resectable tumor condition 1024a, where a tumor 1022a is proximate but not in contact with a lumen 1020. In this condition 1024a, the tumor 1022a may be removed surgically. However, it may not be feasible to remove the tumor in condition 1024b and 1024c, where respectively tumor 1022b is borderline resectable and tumor 1022c is unresectable. In the condition 1024b, tumor 1022b abuts lumen 1020 over a surface of lumen 1020 that is less than 180°. Whereas, in the condition 1024c, tumor 1022c encases lumen 1020 over a surface that is more than 180°. Therefore, for conditions 1024b and 1024c, a catheter arrangement 1026 ablates the tumor 1022b/1022c by delivering vapor into or on the surface of the cellular structure of the tumor. Another catheter arrangement 1028 simultaneously injects a coolant into the lumen 1020 using an injection needle. In another example, cold saline is injected into a blood vessel proximal to a tumor involving the blood vessel while simultaneously ablating the tumor.

Subsequent sections of the present specification describe various applications of the ablation systems and methods of the present specification.

Trans Arterial Vapor Ablation (TAVA) of Tumors

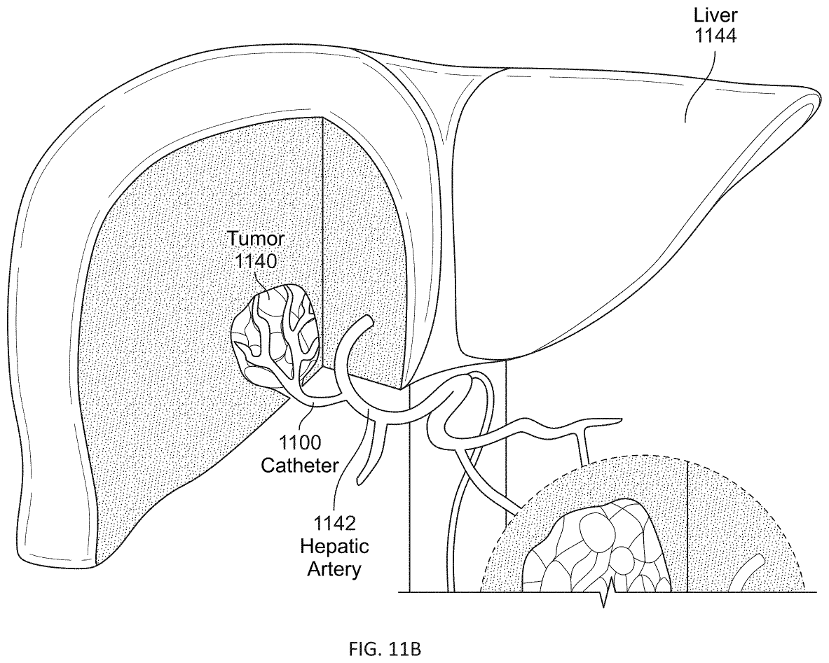

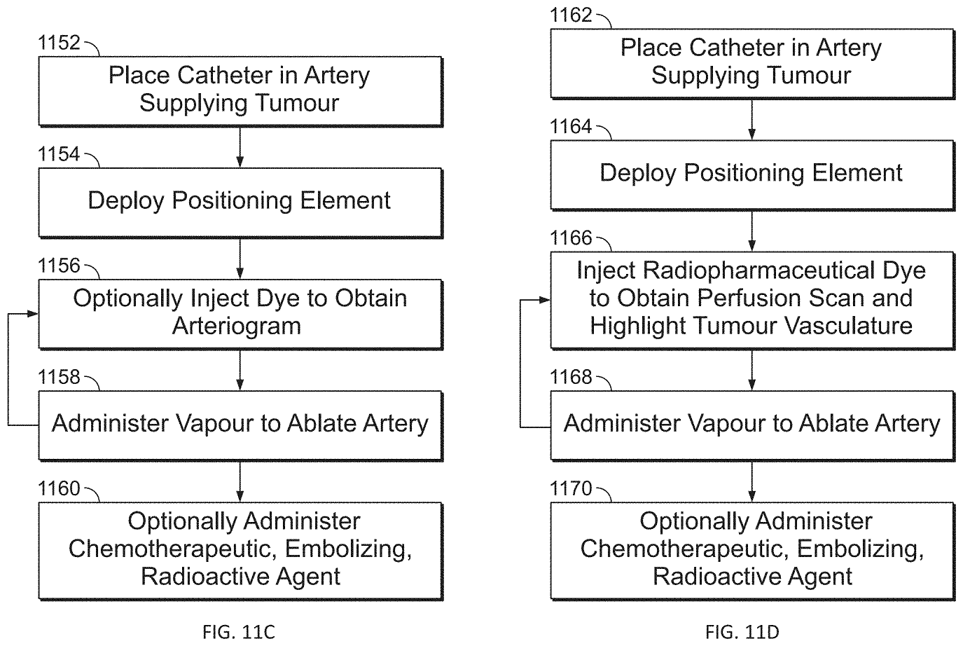

Embodiments of the present specification are used for trans-arterial vapor ablation of tumors. FIG. 11A is a representation of an exemplary catheter arrangement 1100 that is used for vapor ablation of an artery that is supplying blood to a tumor, in accordance with some embodiments of the present specification. FIG. 11B illustrates positioning of the catheter arrangement 1100 of FIG. 11A to treat a tumor 1140 that is present within liver 1144 of a patient, and is fed by hepatic artery 1142, in accordance with some embodiments of the present specification. FIG. 11C is a flow chart illustrating an exemplary method for TAVA of tumors such as tumor 1140 shown in FIG. 11B, using the catheter arrangement 1100 of FIG. 11A.

Referring to FIG. 11A, the catheter arrangement 1100 may correspond to any of the catheter arrangements described in context of the previous figures and embodiments. Specifically, the arrangement 1100 includes a catheter shaft 1102 with a proximal side and a distal side, where the distal side is extended inside a body of the patient. The catheter shaft 1102 comprises an internal heating chamber 1104, disposed within a lumen of the catheter 1102 and configured to heat a fluid provided to the catheter 1102 to change said fluid to a vapor for ablation therapy. The heating chamber 1104 may include an RF electrode array for heating the fluid input from a fluid channel 1122 at the proximal side of the catheter 1102. In one embodiment the fluid is electrically conductive saline and is converted into electrically non-conductive or poorly conductive vapor.

In some embodiments, the catheter 1102 is made of or covered with an insulated material to prevent the escape of ablative energy from the catheter body. An opening 1106 is located proximate the distal side of the catheter 1102 for enabling exit of the vapor or steam generated within the lumen of the catheter 1102. In some embodiments, one or more of associated thermally conductive elements, such as a needles, are extended and deployed or retracted through an opening at the distal end of the catheter 1102, through which the steam exits. During use, cooling fluid such as water, air, or CO₂ is circulated through an optional port to cool the catheter 1102. Vapor for ablation and cooling fluid for cooling are supplied from a port 1122 to the catheter 1102 at its proximal end. An electrical cable 1120 connects a handle 1108 of the catheter 1102 to a power supply and enables operation of multiple electronic controls provided within the handle 1108 to operate the catheter 1102. The various connections and elements of the catheter arrangement 1100 including a microcontroller and functions enabled by the handle 1108 are described in context of FIGS. 1 and 2, and are not repeated here for the sake of brevity. The distal side of the catheter 1102 includes a positioning element 1112 that is configured to expand using a control provided on handle 1108. In some embodiments, the positioning element 1112 is an inflatable balloon that is inflated and deflated using a port 1124. Positioning element 1112 is positioned around the catheter 1102 exterior and acts as a cooling element. The positioning element 1112 is configured to sit at the tissue/air interface such that, as the needle is inserted and heated vapor is directed through the needle to the underlying tissue to be ablated, the positioning element 1112 (which necessarily is cooler) is positioned on the tissue/air interface to help keep the tissue surface at a lower temperature than the underlying tissue being ablated.

Referring simultaneously to FIGS. 11A, 11B, and 11C, at step 1152, catheter arrangement 1100 is positioned within a hepatic artery 1142 that feeds a tumor 1140 in liver 1142 of the patient. At step 1154, positioning element 1112 is deployed so as to occlude the flow of blood through artery 1142 to tumor 1140. The vapor delivery port 1106 is positioned distal from the deployed positioning element 1112. In some embodiments, the positioning element 1112 is an inflatable balloon that is inflated through port 1124 to cause the occlusion of blood flow through the artery 1142. At step 1156, a dye is optionally injected at the position near the distal side of the catheter 1102. In some embodiments, a needle deployed at the distal end of the catheter 1102 includes an opening that allows the dye to be injected. The dye is used to obtain an arteriogram to check placement of the catheter arrangement 1100. At step 1158, an ablative agent, such as vapor or steam, is administered through the vapor delivery port 1106 of the catheter arrangement 1100. The vapor ablates artery 1142 that supplies blood to the tumor 1140. Optionally, the steps of 1156 and 1158 are repeated to obtain arteriogram to check for adequacy of ablation. At step 1160, a chemotherapeutic, an embolizing or a radioactive agent is optionally delivered in conjunction with vapor ablation. While this step is stated separately, it is performed simultaneously with the treatment method of the embodiments of the present specification.

FIG. 11D is a flow chart illustrating another exemplary method for TAVA of tumors such as tumor 1140 shown in FIG. 11B, using the catheter arrangement 1100 of FIG. 11A. Referring simultaneously to FIGS. 11A, 11B, and 11D, at step 1162, catheter arrangement 1100 is positioned within a hepatic artery 1142 that feeds a tumor 1140 in liver 1142 of the patient. At step 1164, positioning element 1112 is deployed so as to occlude the flow of blood through artery 1142 to tumor 1140. The vapor delivery port 1106 is positioned distal from the deployed positioning element 1112. In some embodiments, the positioning element 1112 is an inflatable balloon that is inflated through port 1124 to cause the occlusion of blood flow through the artery 1142. At step 1166, a radiopharmaceutical dye is injected to obtain a perfusion scan of the tumor and to highlight the tumor vasculature. At step 1168, an ablative agent, such as vapor or steam, is administered through the vapor delivery port 1106 of the catheter arrangement 1100. The vapor ablates artery 1142 that supplies blood to the tumor 1140. Optionally, the steps of 1166 and 1168 are repeated to obtain perfusion scan to check for adequacy of ablation. At step 1170, a chemotherapeutic, an embolizing or a radioactive agent is optionally delivered in conjunction with vapor ablation. While this step is stated separately, it is performed simultaneously with the treatment method of the embodiments of the present specification.

The embodiments of FIG. 11A to 11D provide several advantages, which are briefly described here as one or more of the following outcomes: greater than 5% reduction in tumor volume in 6 weeks, greater than 5% reduction in tumor volume maintained for at least 6 weeks, greater than 5% reduction in tumor related mortality in 6 months, greater than 5% reduction in al-cause related mortality in 6 months, greater than 5% tumor-free survival for 6 months, greater than 5% increase in curative resections, greater than 1% reduction in surgical complications with cancer surgery, and greater than 5% decrease in surgical times with cancer surgery.

RF Vapor Neurotomy

Radiofrequency (RF) vapor neurotomy uses heat generated by vapor, using the embodiments of the present specification, to target specific nerves and temporarily turn off their ability to send pain signals. The procedure is also known as radiofrequency vapor ablation. Needles are inserted through the patient's skin near the painful area to deliver the RF vapor to target nerves. Imaging scans may be used during RF vapor neurotomy to ensure that the needles are positioned properly. RF vapor neurotomy can be used for treating pain in the back, neck and buttocks (sacroiliac joint). RF vapor neurotomy is also helpful for treating chronic knee pain and hip joint pain.

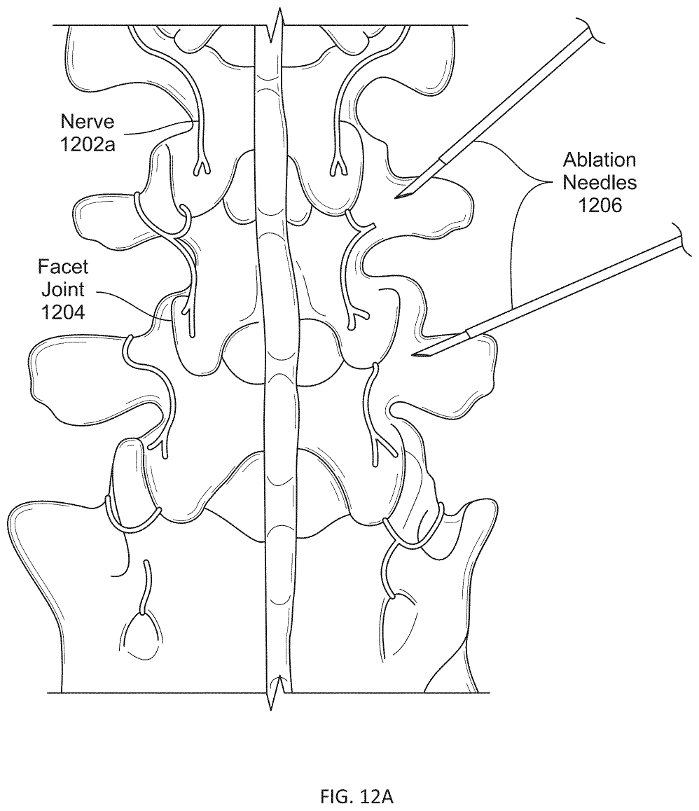

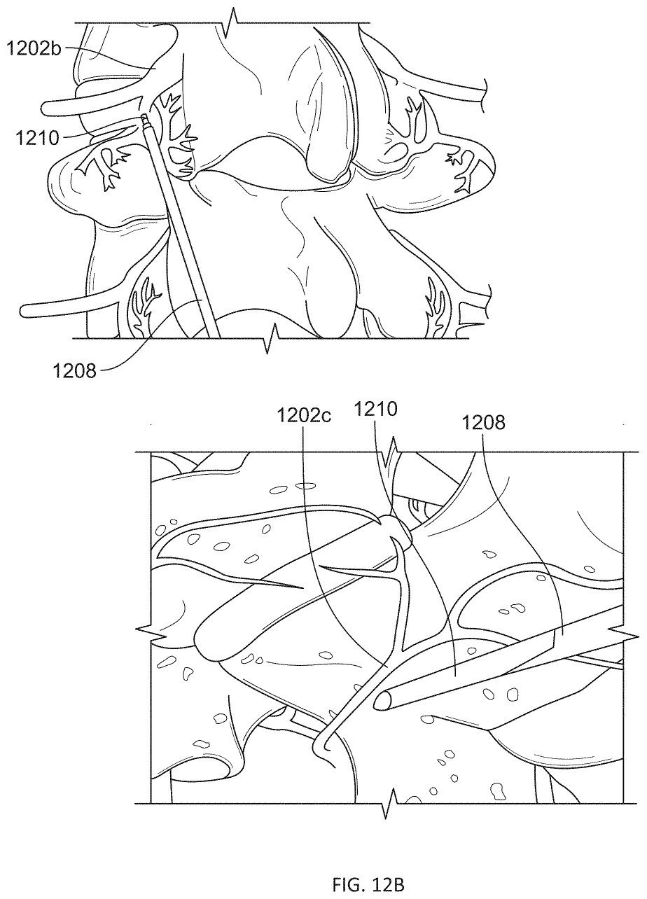

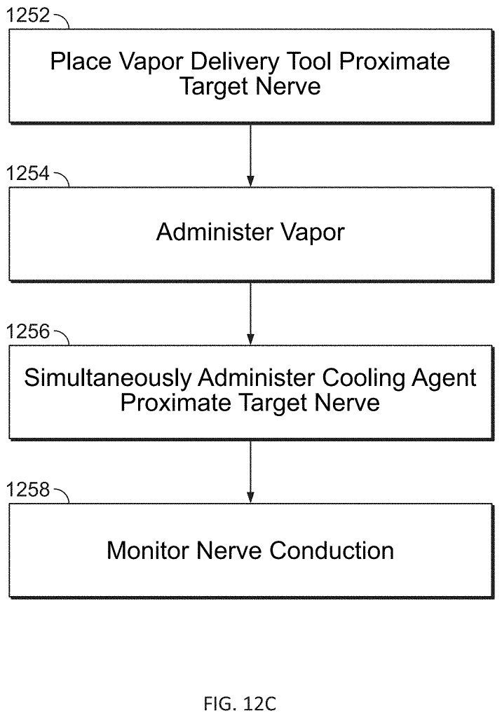

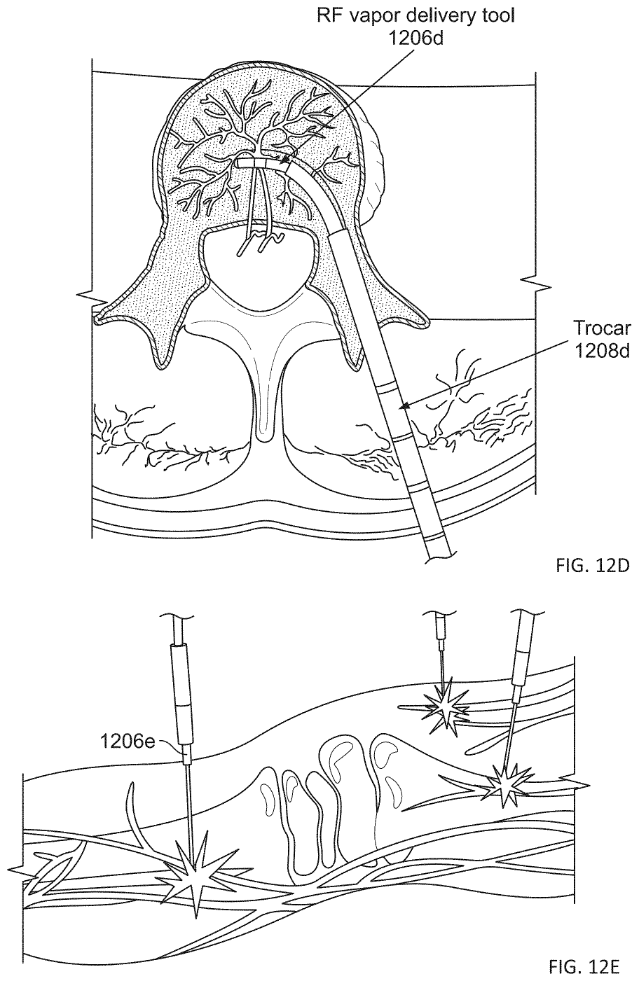

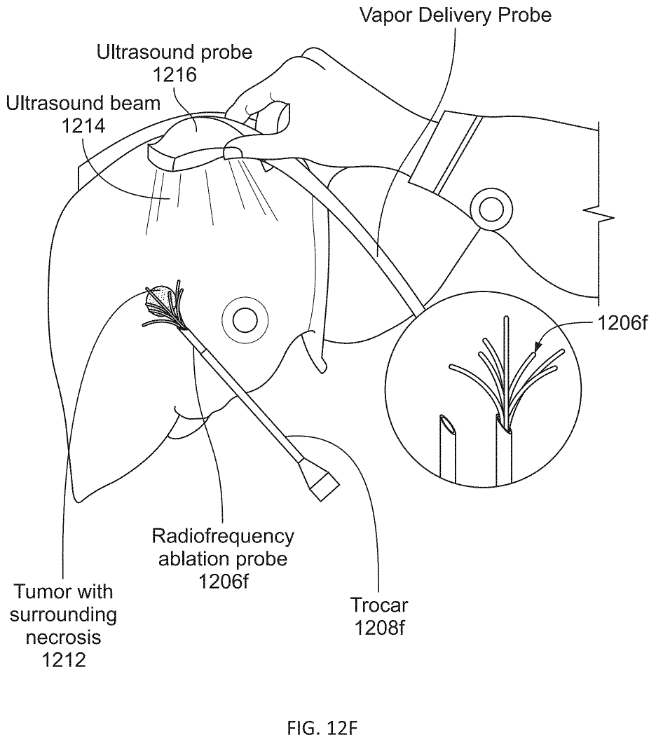

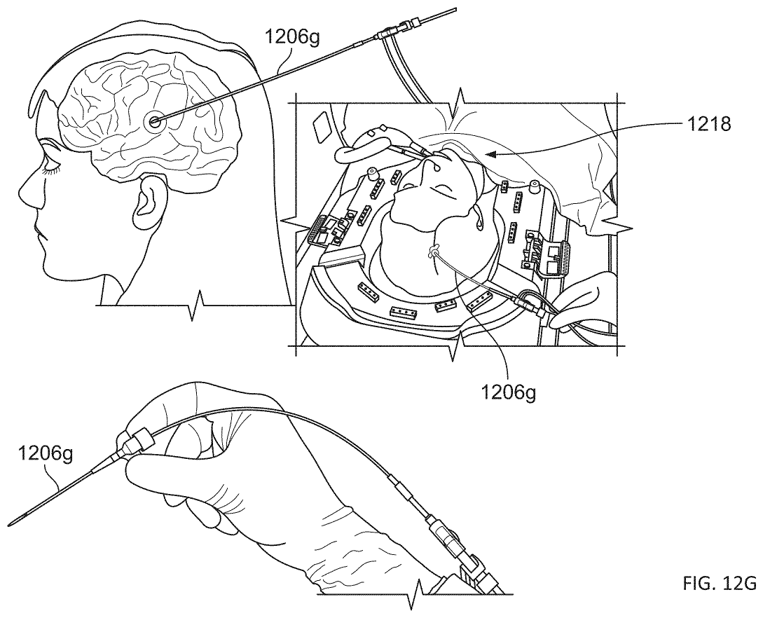

FIG. 12A illustrates using multiple vapor ablation tools 1206 to treat pain transmitted by a nerve 1202a proximate a facet joint 1204 in a spinal motion segment of a patient, in accordance with some embodiments of the present specification. FIG. 12B illustrates using trocar needles 1208 for administering vapor ablation using ablation tools 1210 to treat pain transmitted by nerves 1202b and 1202c in different parts of a patient's body, in accordance with some embodiments of the present specification. FIG. 12C is a flow chart illustrating an exemplary process for treating pain using RF vapor neurotomy, in accordance with the present specification. Referring simultaneously to FIGS. 12A, 12B, and 12C, at step 1252, a vapor ablation tool 1206/1210 is placed proximate to the target nerve. The target nerve is the nerve that is responsible for causing or conducting the pain. The ablation tool 1206/1210 may be any one of the tools described previously in context of FIGS. 1 to 8E. The ablation tool 1206 is delivered through a catheter arrangement or an endoscope, while tools 1210 are delivered through trocar needles. In some embodiments, imaging methods are used to locate the tip of the vapor delivery tool 1206/1210 proximate the target nerve. At step 1254, vapor is delivered through the tool 1206/1210 to ablate the target nerve so as to permanently damage the nerve that is responsible for the sensation of pain. In some embodiments, the vapor ablation tool 1206/1210 includes a plurality of ports to deliver ablation vapor. The vapor, in some embodiments, is delivered at a pressure less than 5 atm and temperature less than 110° C. In some embodiments, the vapor is delivered for a period of less than 10 seconds in each application. At step 1256, simultaneous to step 1254, a cooling agent such as saline is administered proximate the target nerve to prevent damage from ablative vapor to other areas near the proximate nerve. In some embodiments, the cooling agent is passed through a lumen in the trocar 1208 or the endoscope between the vapor delivery tool 1210 and the wall of the trocar 1208, thereby cooling the wall of the trocar 1208 (or endoscope) to prevent damage to the adjacent structures from the vapor delivery tool 1210. Any excess vapor is allowed to vent out between the tool 1210 and the trocar 1208. At step 1258, nerve conduction by the target nerve is monitored continually during the application of vapor, and the vapor delivery is stopped once the nerve conduction is halted.

Other Applications of RF Vapor Ablation

FIG. 12D illustrates use of a vapor delivery tool 1206d to administer vapor for basivertebral nerve ablation using the RF vapor ablation procedure of FIG. 12C. The figure shows use of a trocar 1208d to position the tool 1206d for administering the vapor. FIG. 12E illustrates use of a vapor delivery tool 1206e with a needle to administer vapor for treating arthritis pain using the RF vapor ablation procedure of FIG. 12C. FIG. 12F illustrates use of the RF vapor ablation procedure of FIG. 12C to treat a tumor 1212 in the liver, in accordance with some embodiments of the present specification. An RF vapor ablation delivery tool 1206f is positioned proximate the tumor 1212 using a trocar 1208f to deliver ablative vapor. In some embodiments, multiple probes or needles are used by the tool 1206f to administer the vapor for ablation. Ultrasound beam 1214 from an ultrasound probe 1216 may be used simultaneously, to guide placing of the tool 1206f proximate to the tumor 1212.

FIG. 12G illustrates MRI guided use of a vapor delivery tool 1206g to treat a focal lesion in the brain using the RF vapor ablation procedure of FIG. 12C, in accordance with some embodiments of the present specification. A patient 1218 with a focal lesion in the brain causing a focal neurological deficit or seizure activity is treated by inserting vapor delivery tool 1206g through a burr hole in the skull through the brain to a focal brain soft tissue lesion using stereotactic guidance for precise vapor delivery tool 1206g placement. Imaging such as an MRI is performed to verify the location of the vapor delivery tool 1206g. Real time MRI thermography or image thermography is used to initiate and control the vapor thermal energy delivery for coagulation of the focal neurological lesion. In some embodiments, pressure of the vapor delivery is monitored so as to maintain the pressure below 5 atm. Treatment using embodiments of the present specification decreases size of the focal lesion by at least 10%. Additionally, the seizure frequency, intensity or duration in the patient decreases by 10%.

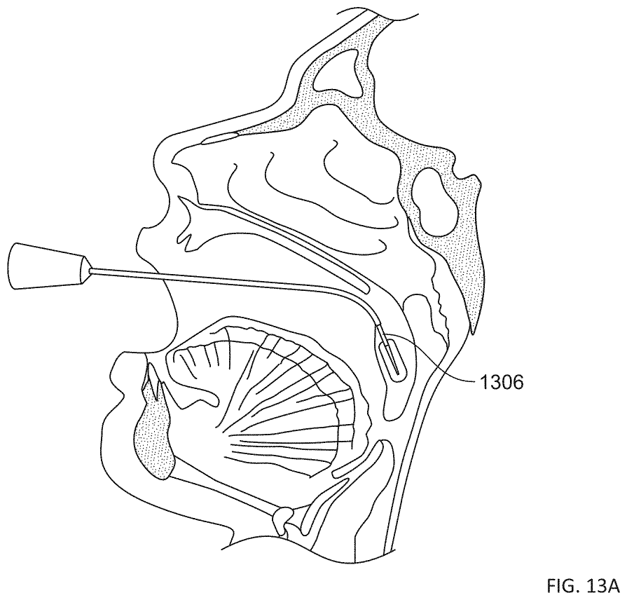

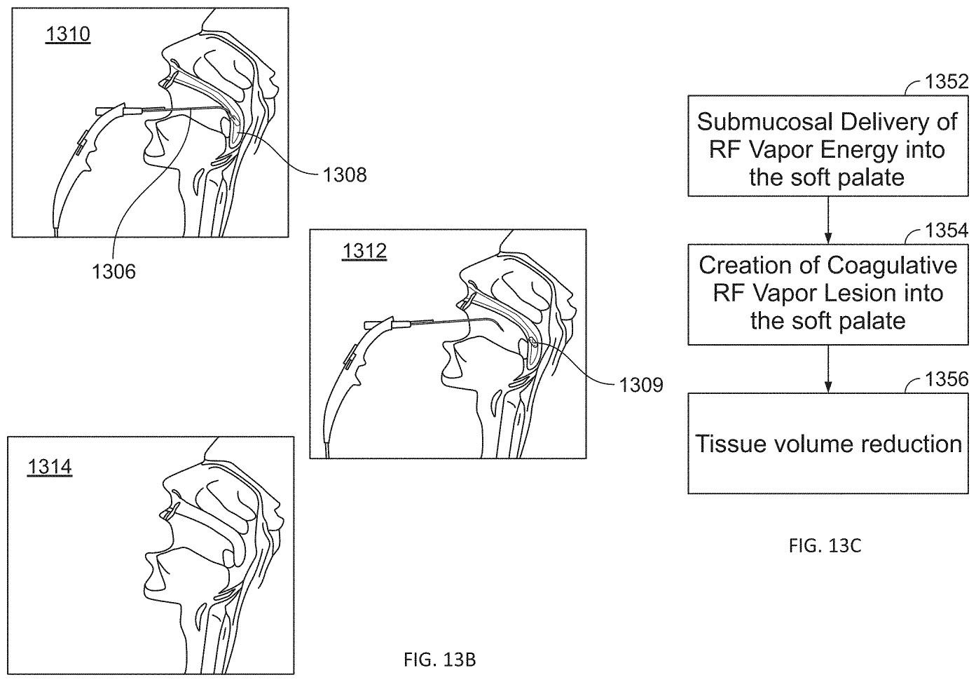

FIG. 13A illustrates use of a vapor delivery tool 1306 to treat sleep apnea using the RF vapor ablation procedure, in accordance with some embodiments of the present specification. The RF vapor ablation technique uses very low energy to create finely controlled coagulative zones underneath the mucosal layer. These zones are naturally resorbed by the body, altering the tissue structure by reducing excess tissue. RF vapor ablation is a minimally invasive, outpatient procedure which reduces and tightens excess tissue in the upper airway responsible for Obstructive Sleep Apnea Syndrome, including the base of tongue which is the most difficult to treat source of the obstruction. The commonly outpatient procedure usually takes place under local anesthesia, with the patient typically resuming normal activities the following day. Over a period of three to twelve weeks the treated tissue is reabsorbed, leading to volume reduction and improves airway obstruction. The procedure itself typically takes less than 30 minutes, with less than 5 minutes of RF vapor delivery. Mucosal Surface temperature can be monitored to guide the duration and delivery of the RF vapor energy. Alternatively, mucosa could be cooled to prevent thermal damage to the mucosal layer from the RF vapor energy. More than one treatment may be needed for some patients to achieve optimal results.

FIG. 13B illustrates the steps involved in RF vapor ablation of palate to treat sleep apnea using the ablation systems and methods in accordance with the embodiments of the present specification. FIG. 13C is a flow chart illustrating the steps involved in RF vapor ablation of palate to treat sleep apnea using the ablation systems and methods in accordance with the embodiments of the present specification. Referring simultaneously to FIGS. 13B and 13C, at step 1352, RF vapor energy is delivered in to the soft palate of a patient. RF vapor delivery tool 1306 in inserted through the mouth of the patient to reach and ablate the soft palate tissue 1308, as shown in view 1310. The patient is fully awake throughout the treatment. The physician first applies a local anesthetic to the uvula and palate, similar to that used in a dental procedure. A few minutes later the RF vapor device 1306, which is connected to a radiofrequency vapor generator, is placed into the mouth. A vapor delivery port located at the distal end of the device 1306 is inserted into the soft palate 1308. RF vapor is delivered through the vapor delivery port. Part of the vapor delivery device 1306 is insulated to protect the delicate surface of the tissue 1308. Through controlled delivery of RF vapor energy, the tissue 1308 is heated in a limited area around the vapor delivery port. At step 1354, corresponding to view 1312, the RF vapor ablation procedure of the present specification creates a submucosal lesion 1309 in the soft palate. The patients may experience some swelling and have a mild sore throat. Following the procedure, a patient may take an over-the-counter analgesic for one to three days. At step 1356, seen in view 1314, the lesion is naturally resorbed by the body over a period of three to six weeks, leading to tissue volume reduction. In addition, the collagen in the treated area tends to contract, lifting the uvula, stiffening the tissue and reducing its propensity to vibrate. With the reduction and tightening of the obstructive tissue, snoring is reduced in many patients.

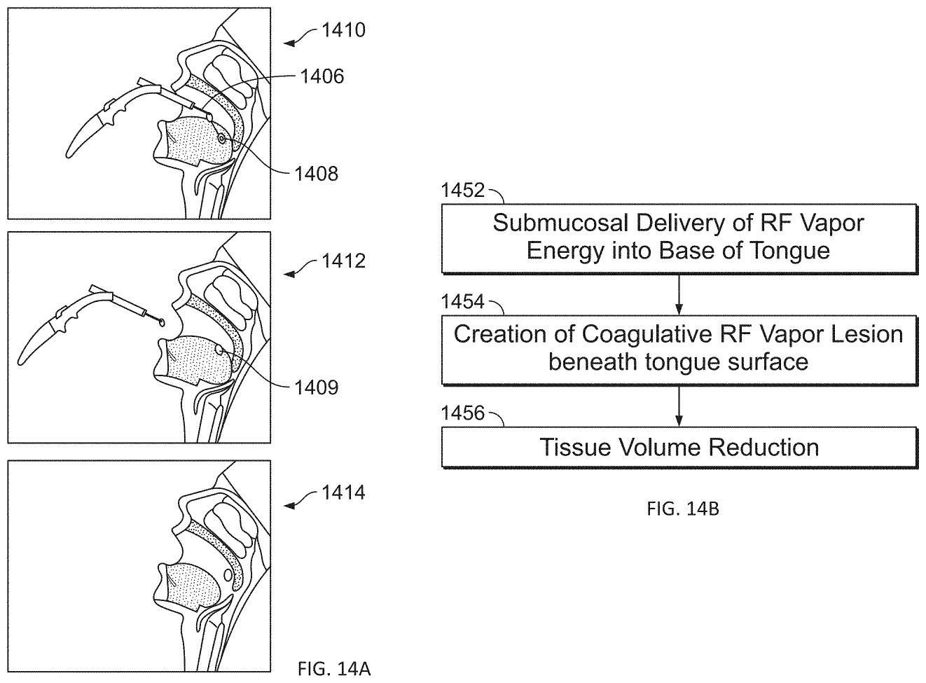

FIG. 14A illustrates the steps involved in RF vapor ablation of tongue to treat obstructive sleep apnea using the ablation systems and methods in accordance with the embodiments of the present specification. FIG. 14B is a flow chart illustrating the steps involved in RF vapor ablation of tongue to treat obstructive sleep apnea using the ablation systems and methods in accordance with the embodiments of the present specification. Referring simultaneously to FIGS. 14A and 14B, at step 1452, RF vapor energy is delivered beneath the surface tissue of base of tongue. RF vapor delivery tool 1406 is inserted through the mouth of the patient to reach the base of tongue. A physician inserts a surgical hand piece needle electrode into the base of the tongue. An RF generator delivers energy to ablate tissue 1408 beneath surface of the base of tongue, as shown in view 1410. The procedure may take place in an outpatient setting under local anesthesia. Through controlled delivery of RF vapor energy, the tissue 1408 is heated in a limited area around the needle electrode. At step 1454, corresponding to view 1412, the RF vapor ablation procedure of the present specification creates a coagulative lesion 1409 beneath the surface. Discomfort is minimal during the procedure and the surface tissue is protected from thermal damage. Over the course of one or more procedures, one or a number of lesions may be created in the base of tongue. At step 1456, seen in view 1414, the lesion is naturally resorbed by the body over a period of three to eight weeks, leading to tissue volume reduction, and helping to open the airway during sleep.

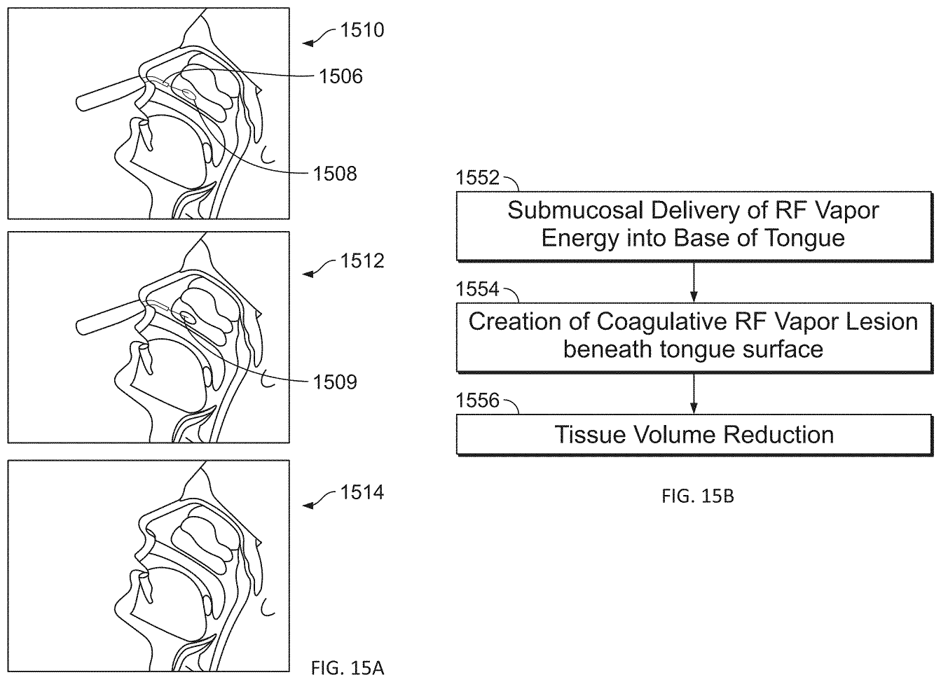

FIG. 15A illustrates the steps involved in RF vapor ablation of inferior turbinate in the submucosal space to relieve chronic nasal obstruction using the ablation systems and methods in accordance with the embodiments of the present specification. FIG. 15B is a flow chart illustrating the steps involved in RF vapor ablation of inferior turbinate in the submucosal space to relieve chronic nasal obstruction using the ablation systems and methods in accordance with the embodiments of the present specification. Referring simultaneously to FIGS. 15A and 15B, at step 1552, RF vapor energy is delivered beneath the mucosa into the submucosal tissue. RF vapor delivery tool 1506 is inserted into the inferior turbinate and one of the vapor delivery ports is positioned in the submucosal space 1508. A physician may use direct vision or endoscopic guidance to insert and position the vapor delivery ports. The mucosal temperature is optionally monitored to direct the delivery of RF vapor energy. Alternatively, mucosal surface is actively cooled to prevent significant thermal injury to the nasal mucosa. The procedure may take place in an outpatient setting under local anesthesia. Through controlled delivery of RF vapor energy, tissue in the submucosal space 1508 is heated in a limited area around the vapor delivery port. At step 1554, corresponding to view 1512, the RF vapor ablation procedure of the present specification creates a coagulative lesion 1509. At step 1556, seen in view 1514, the lesion is naturally resorbed by the body, leading to tissue volume reduction, and relieving nasal obstruction. Embodiments of FIGS. 15A and 15B provide an effective treatment for patients who suffer from chronic turbinate hypertrophy enlargement.

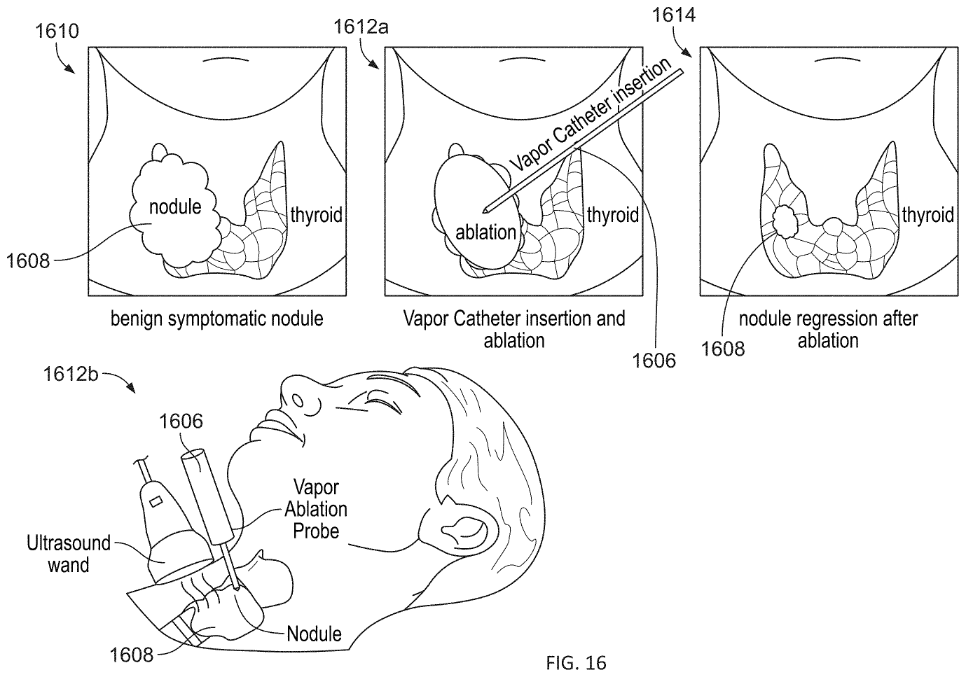

FIG. 16 illustrates the steps involved in RF vapor ablation of a solitary thyroid nodule to improve thyroid function, using the ablation systems and methods in accordance with the embodiments of the present specification. The solitary thyroid nodule may be of a volume that is less than or equal to 25 ml. A view 1610 illustrates a benign symptomatic thyroid nodule in a patient. Views 1612a and 1612b illustrate insertion of a RF vapor delivery tool 1606 that is inserted by a physician preferably under imaging guidance, such as under the guidance of an Ultrasound probe. One or more ports of vapor ablation are inserted inside the thyroid nodule 1608 to ablate the nodule. An RF generator delivers controlled RF energy to ablate a limited area around the vapor ablation ports. The ablation is performed without significantly ablating the surrounding normal thyroid tissue. Using the embodiments of the present specification, thyroid function may be improved by at least 10% in about 6 months from the time of the treatment. View 1614 illustrates regression of thyroid nodule 1608 after ablation. Embodiments of the present specification may normalize thyroid function in 10% of medium size AFTN and in more than 15% of small size AFTN at six months after the treatment. Nodule volume reduction of >20% can be achieved between six and 24 months from the time of treatment.

The above examples are merely illustrative of the many applications of the system of the present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A method for selectively ablating at least one of a target tissue area of a patient, the method comprising:
providing an ablation system comprising:
at least one pump;
a coaxial catheter for inserting into the patient, the coaxial catheter comprising:
an outer catheter for advancing to the target tissue of the patient;
an inner catheter for advancing into the target tissue of the patient, concentric and slidable within the outer catheter, wherein the inner catheter is in fluid communication through a catheter connection port with the at least one pump, wherein a proximal end of the inner catheter is connected to the catheter connection port to place the inner catheter in fluid communication with the at least one pump, wherein the inner catheter comprises:
at least one lumen to transport an ablative agent delivered from the at least one pump;
at least one electrode positioned within the at least one lumen;
at least one positioning element along a length of the inner catheter; and
at least one opening proximate to the positioning element of the inner catheter;
a controller having at least one processor in data communication with the at least one pump, wherein, upon activating, the controller is configured to:
control the delivery of the ablative agent into the at least one lumen in the coaxial catheter;
control the delivery of an electrical current to the at least one electrode positioned within the at least one lumen of the inner catheter; and
control vapor generated from the ablative agent;
inserting the coaxial catheter into the target tissue of the patient;
applying the positioning element proximate the target tissue area enclosing at least a portion of the target tissue; and
programming the controller to control a delivery of the vapor such that the target tissue is ablated to cause differential damage to different cellular components in the target tissue.

2. The method of claim 1 wherein the at least one positioning element is advanced until the distal end of the positioning element encloses the target tissue area.

3. The method of claim 1 wherein the at least one positioning element is advanced until the distal end of the positioning element is proximate the target tissue area.

4. The method of claim 1 wherein programming the controller to control a delivery of the vapor such that the target tissue is ablated to cause differential damage comprises damaging more cellular structure relative to extra cellular matrix (ECM).

5. The method of claim 4 wherein the target tissue is ablated for a time period at a temperature of up to 60° C.

6. The method of claim 4 wherein greater than 50% of the cellular structure undergoes irreversible damage and less than 50% of the ECM is damaged.

7. The method of claim 1 wherein programming the controller comprises maintaining pressure at the target tissue area less than 5 atm.

8. The method of claim 1 wherein programming the controller comprises delivering the vapor at a temperature between 99° C. and 110° C.

9. The method of claim 1 wherein programming the controller comprises delivering the vapor of a quality greater than 25%.

10. The method of claim 1 wherein programming the controller to control a delivery of the vapor such that the target tissue is ablated to cause differential damage comprises damaging more cellular structure relative of tumor relative to normal cellular structure.

11. The method of claim 1 further comprising treating a tumor proximate one of a blood vessel and a bowel wall.

12. The method of claim 1 further comprising performing trans-arterial vapor ablation of tumors.

13. The method of claim 12 comprising providing the ablation system positioned within a hepatic artery that feeds a tumor in a liver.

14. The method of claim 1 further comprising treating pain in at least one of a back, a neck, a sacroiliac joint, a knee pain, and a hip joint.

15. The method of claim 14 comprising treating pain transmitted by a nerve proximate a facet joint in a spinal motion segment of a patient.

16. The method of claim 14 comprising administering vapor for basivertebral nerve ablation.

17. The method of claim 1 comprising treating arthritis pain.

18. The method of claim 1 comprising treating a focal lesion in the brain.

19. The method of claim 1 comprising treating sleep apnea by at least one of ablation of a palate and ablation of a tongue.

20. The method of claim 1 comprising ablating an inferior turbinate in a submucosal space to relieve chronic nasal obstruction.

21. The method of claim 1 comprising ablating a solitary thyroid nodule to improve thyroid function.
